(19) United States
(12) Patent Application Publication
(10) Pub. No.: US 2025/0270506 A1
(43) Pub. Date: Aug. 28, 2025

(54) T Cells

(71) Applicant: Instituto de Medicina Molecular João Lobo Antunes, Lisbon (PT)

(72) Inventors: Cristina DA CONCEIÇÃO VARANDAS FERREIRA, Lisbon (PT); Marc VELDHOEN, Lisbon (PT)

(73) Assignee: Instituto de Medicina Molecular João Lobo Antunes, Lisbon (PT)

(21) Appl. No.: 17/999,087

(22) PCT Filed: May 13, 2021
(86) PCT No.: PCT/PT2021/050013

§ 371 Date: Nov. 17, 2022

(30) Foreign Application Priority Data
May 18, 2020 (GB) 202007321

(51) Int. Cl. C12N 5/0783; A61K 35/17

(52) U.S. Cl. CPC C12N 5/0636; A61K 35/17; C12N 2501/15; C12N 2501/2302; C12N 2501/2307; C12N 2501/2315; C12N 2502/1114

(57) ABSTRACT

The invention relates to T cells, and to methods of producing tissue-resident memory T cells (T_{RM}). The invention concerns tissue-resident memory T cells (T_{RM}) per se which have been obtained from the methods of the invention, compositions comprising these T_{RM} cells, and the use of these T_{RM} cells and the compositions in therapy, such as in immuno-therapy for treating cancer.

The invention relates to T cells, and, particularly, although not exclusively, to methods of producing tissue-resident memory T cells (T_{RM}), tissue-resident memory T cells (T_{RM}) per se which have been obtained from the methods of the invention, compositions comprising these T_{RM} cells, and the use of these T_{RM} cells and the compositions in therapy, such as immuno-therapy for treating cancer.

### REFERENCE TO SEQUENCE LISTING

This application contains a Sequence Listing submitted as .txt file named “Sequence listing_ST25”, having a size in bytes of 114 kilobytes, and created on May 7, 2021. The information contained in this electronic file is hereby incorporated by reference in its entirety.

Immunotherapy using immune checkpoint inhibitors, such as blocking antibodies against PD-1 and CTLA-4, has significantly promoted cancer-free survival. Importantly, adoptive transfers, such as with chimeric antigen receptor (CAR) T cells [1], general tumour detecting delta-one γδ T cells (DOT) [2, 3] or MR-1 restricted T cells [4], have achieved very promising results. However, questions remain whether these approaches will be sufficiently effective against solid tumours, such as breast cancer, in which durable responses are only obtained in a fraction of patients. The success of T cell immunotherapy, especially in solid tumours, hinges on delivering and activating tumour-specific lymphocytes with cytotoxic activity, such as CD8+ T cells, within tumour tissues.

Breakthroughs in tissue immunity have revealed the existence of tissue-resident memory CD8+ T (T_{RM}) cells [5, 6], which can penetrate deeply into tissues. Recent years have shown a strong correlation between the presence of T_{RM} cells in tumour, identified by their expression of the marker CD103, and positive prognosis in patients. T_{RM} cells strongly correlate, better than total CD8+ T cell numbers, with increased overall survival and extended period of disease-free state in solid tumours such as breast, lung, ovarian and cervical cancer [7-15]. Indeed, T_{RM} cell directly link to empowered cytotoxic T cell response in human solid tumours [16, 17].

Thus, exploring improved mechanisms for immunotherapy, with emphasis in producing and delivering cytotoxic CD8+ T cells deep into tissues is of great importance and an essential step change to successful cell-based cancer immunotherapy against solid tumours.

The inventors hypothesised that T regulatory (T_{REG}) cells are important in the generation of T cells that are able to penetrate deeply into tissues and that are highly effective against solid tumours. The inventors have been able to delineate and identify the factors required to generate T_{RM} cells to enable the generation of T_{RM} cells in vitro with the aim of generating anti-tumour T cells with tissue penetrating properties.

Accordingly, in a first aspect of the invention, there is provided a method for producing a tissue-resident memory T cell (T_{RM}), the method comprising culturing a lymphocyte in the presence of transforming growth factor beta (TGFβ) and/or co-culturing the lymphocyte with a regulatory T cell.

Advantageously, as described in the Examples, the inventors have developed a protocol to generate T cells for use in cell therapy by establishing the in vitro requirements required for the development of tissue-penetrating T cells, i.e. tissue-resident memory T cell (T_{RM}). The production of such cells will result in the production of T_{RM} cells that enable delivery and activation of disease-specific lymphocytes with cytotoxic activity within diseased tissues, such as tumour tissue, and also metastasising tumours, thereby significantly broadening the therapeutic tool-kit for T cell based therapies. Furthermore, as described in the Examples, the inventors have developed a novel protocol to generate T_{RM} cells for use in cell therapy, without including T_{REG} cells in the culture.

Preferably, the method is performed ex vivo or in vitro.

Preferably, the method comprises culturing the lymphocyte in the presence of TGFβ. Preferably, in some embodiments, the method does not comprise culturing the lymphocyte in the presence of regulatory T cells.

Preferably, the TGFβ is bioactive (i.e. activated). Preferably, the TGFβ is mammalian. The TGFβ may be rodent, dog, horse or pig TGFβ. The rodent may be a rat or a mouse. Most preferably, the TGFβ is human TGFβ.

In one embodiment, TGFβ may be TGFβ₁ represented by Genebank ID No: 7040, which is provided herein as SEQ ID No: 1, as follows:

[SEQ ID No: 1]
MPPSGLRLLPLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFFNTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQSSRHRRALDTNYCFSSTEKNCC
VRQLYIDFRKDLGWKWIHEPKGYHANFCLGPCPYIWSLDTQYSKVLALY
NQHNPGASAAPCCVPQALEPLPIVYYVGRKPKVEQLSNMIVRSCKCS

Thus, preferably TGFβ comprises or consists of a sequence as substantially set out in SEQ ID No: 1, or a fragment or variant thereof.

In one embodiment, TGFβ may be TGFβ₂ represented by Genebank ID No: 7042, which is provided herein as SEQ ID No: 14, as follows:

[SEQ ID No: 14]
MHYCVLSAFLILHLVTVALSLSTCSTLDMDQFMRKRIEAIRGQILSKLK
LTSPPEDYPEPEEVPPEVISIYNSTRDLLQEKASRRAAACERERSDEEY
YAKEVYKIDMPPFFPSENAIPPTFYRPYFRIVRFDVSAMEKNASNLVKA
EFRVFRLQNPKARVPEQRIELYQILKSKDLTSPTQRYIDSKVVKTRAEG
EWLSFDVTDAVHEWLHHKDRNLGFKISLHCPCCTFVPSNNYIIPNKSEE
LEARFAGIDGTSTYTSGDQKTIKSTRKKNSGKTPHLLLMLLPSYRLESQ
QTNRRKKRALDAAYCFRNVQDNCCLRPLYIDFKRDLGWKWIHEPKGYNA
NFCAGACPYLWSSDTQHSRVLSLYNTINPEASASPCCVSQDLEPLTILY
YIGKTPKIEQLSNMIVKSCKCS

Thus, preferably TGFβ comprises or consists of a sequence as substantially set out in SEQ ID No: 14, or a fragment or variant thereof.

In one embodiment, TGFβ may be TGFβ₃ represented by Genebank ID No: 7043, which is provided herein as SEQ ID No: 16, as follows:

[SEQ ID No: 16]
MKMHLQRALVVLALLNFATVSLSLSTCTTLDFGHIKKKRVEAIRGQILS
KLRLTSPPEPTVMTHVPYQVLALYNSTRELLEEMHGEREEGCTQENTES
EYYAKEIHKFDMIQGLAEHNELAVCPKGITSKVFRFNVSSVEKNRTNLF
RAEFRVLRVPNPSSKRNEQRIELFQILRPDEHIAKQRYIGGKNLPTRGT
AEWLSFDVTDTVREWLLRRESNLGLEISIHCPCHTFQPNGDILENIHEV
MEIKFKGVDNEDDHGRGDLGRLKKQKDHHNPHLILMMIPPHRLDNPGQG
GQRKKRALDTNYCFRNLEENCCVRPLYIDFRQDLGWKWVHEPKGYYANF
CSGPCPYLRSADTTHSTVLGLYNTLNPEASASPCCVPQDLEPLTILYYV
GRTPKVEQLSNMVVKSCKCS

Thus, preferably TGFβ comprises or consists of a sequence as substantially set out in SEQ ID No: 16, or a fragment or variant thereof.

Preferably, TGFβ is present at a concentration of between 0.01 ng/ml and 50 ng/ml. More preferably, the TGFβ may be present at a concentration of between 0.1 ng/ml and 20 ng/ml, or between 0.1 ng/ml and 10 ng/ml. Most preferably, the TGFβ may be present at a concentration of between 0.25 ng/ml and 5 ng/ml, and more preferably between 0.5 ng/ml and 5 ng/ml.

Preferably, the lymphocyte is a naïve, effector or memory CD8+ T lymphocyte.

Preferably, the lymphocyte is a naïve or effector CD8+ T lymphocyte.

Preferably, the lymphocyte is a naïve CD8+ T lymphocyte. When the lymphocyte is human, the naïve CD8+ T lymphocyte may be defined by expression of cluster of differentiation 45 isoform RA (CD45RA+), C-C chemokine receptor type 7 (CCR7+) and/or cluster of differentiation 27 (CD27+). The naïve CD8+ T lymphocyte may be further characterised by lack of expression of cluster of differentiation 45 isoform RO (CD45RO−).

When the lymphocyte is murine, preferably mouse, the naïve CD8+ T lymphocyte may be defined by expression of cluster of differentiation 67 isoform L (CD67L+), C-C chemokine receptor type 7 (CCR7+), cluster of differentiation 127 (CD127+) and/or cluster of differentiation 27 (CD27+). The naïve CD8+ T lymphocyte may be further defined by low levels of expression of cluster of differentiation 44 (CD44+).

Preferably, the lymphocyte is an effector CD8+ T-lymphocyte. When the lymphocyte is human, the effector CD8+ T lymphocyte may be characterised by expression of cluster of differentiation 45 isoform RA (CD45RA+) and/or cluster of differentiation 45 isoform RO (CD45RO+). The effector CD8+ T lymphocyte may be further characterised by lack of expression of C-C chemokine receptor type 7 (CCR7−).

When the lymphocyte is murine, preferably mouse, the effector CD8+ T lymphocyte may be characterised by high levels of expression of cluster of differentiation 44 (CD44+) and/or absence of expression of cluster of differentiation 62 ligand (CD62L).

Preferably, the lymphocyte is a memory CD8+ lymphocyte. The memory CD8+ T lymphocyte may be a central memory CD8+ T lymphocyte or an effector memory CD8+ T lymphocyte.

When the lymphocyte is human, the central memory CD8+ T lymphocyte may be characterised by expression of cluster of differentiation 45 isoform RO (CD45RO+). The central memory CD8+ T lymphocyte may be further characterised by lack of expression of cluster of differentiation 45 isoform RA (CD45RA−), C-C chemokine receptor type 7 (CCR7−) cluster of differentiation 27 (CD27−) and/or cluster of differentiation 62L (CD62L−).

When the lymphocyte is murine, preferably mouse, the central memory CD8+ T lymphocyte may be characterised by high levels of expression of cluster of differentiation 44 (CD44+) and/or expression of cluster of differentiation 62 Ligand (CD62L).

When the lymphocyte is human, the effector memory CD8+ T lymphocyte may be characterised by expression of cluster of differentiation 45 isoform RO (CD45RO+). The effector memory CD8+ T lymphocyte may be further characterised by lack of expression of cluster of differentiation 45 isoform RA (CD45RA−), C-C chemokine receptor type 7 (CCR7−) cluster of differentiation 27 (CD27−) and/or cluster of differentiation 62L (CD62L−).

When the lymphocyte is murine, preferably mouse, the effector memory CD8+ T lymphocyte may be characterised by high levels of expression of cluster of differentiation 44 (CD44+) and/or no expression of cluster of differentiation 62 ligand (CD62L).

In one embodiment, CD45RA may be represented by Genebank ID No: 5788, which is provided herein as SEQ ID No: 18, as follows:

[SEQ ID No: 18]
MTMYLWLKLLAFGFAFLDTEVFVTGQSPTPSPTGLTTAKMPSVPLSSDPLPTHTTAFSPASTFERENDFS
ETTTSLSPDNTSTQVSPDSLDNASAFNTTGVSSVQTPHLPTHADSQTPSAGTDTQTFSGSAANAKLNPTP
GSNAISDVPGERSTASTFPTDPVSPLTTTLSLAHHSSAALPARTSNTTITANTSDAYLNASETTTLSPSG
SAVISTTTIATTPSKPTCDEKYANITVDYLYNKETKLFTAKLNVNENVECGNNTCTNNEVHNLTECKNAS
VSISHNSCTAPDKTLILDVPPGVEKFQLHDCTQVEKADTTICLKWKNIETFTCDTQNITYRFQCGNMIFD
NKEIKLENLEPEHEYKCDSEILYNNHKFTNASKIIKTDFGSPGEPQIIFCRSEAAHQGVITWNPPQRSFH
NFTLCYIKETEKDCLNLDKNLIKYDLQNLKPYTKYVLSLHAYIIAKVQRNGSAAMCHFTTKSAPPSQVWN
MTVSMTSDNSMHVKCRPPRDRNGPHERYHLEVEAGNTLVRNESHKNCDFRVKDLQYSTDYTFKAYFHNGD
YPGEPFILHHSTSYNSKALIAFLAFLIIVTSIALLVVLYKIYDLHKKRSCNLDEQQELVERDDEKQLMNV
EPIHADILLETYKRKIADEGRLFLAEFQSIPRVFSKFPIKEARKPFNQNKNRYVDILPYDYNRVELSEIN
GDAGSNYINASYIDGFKEPRKYIAAQGPRDETVDDFWRMIWEQKATVIVMVTRCEEGNRNKCAEYWPSME
EGTRAFGDVVVKINQHKRCPDYIIQKLNIVNKKEKATGREVTHIQFTSWPDHGVPEDPHLLLKLRRRVNA
FSNFFSGPIVVHCSAGVGRTGTYIGIDAMLEGLEAENKVDVYGYVVKLRRQRCLMVQVEAQYILIHQALV
EYNQFGETEVNLSELHPYLHNMKKRDPPSEPSPLEAEFQRLPSYRSWRTQHIGNQEENKSKNRNSNVIPY
DYNRVPLKHELEMSKESEHDSDESSDDDSDSEEPSKYINASFIMSYWKPEVMIAAQGPLKETIGDFWQMI
FQRKVKVIVMLTELKHGDQEICAQYWGEGKQTYGDIEVDLKDTDKSSTYTLRVFELRHSKRKDSRTVYQY
QYTNWSVEQLPAEPKELISMIQVVKQKLPQKNSSEGNKHHKSTPLLIHCRDGSQQTGIFCALLNLLESAE
TEEVVDIFQVVKALRKARPGMVSTFEQYQFLYDVIASTYPAQNGQVKKNNHQEDKIEFDNEVDKVKQDAN
CVNPLGAPEKLPEAKEQAEGSEPTSGTEGPEHSVNGPASPALNQGS

Thus, preferably CD45RA comprises or consists of a sequence as substantially set out in SEQ ID No: 18, or a fragment or variant thereof.

In one embodiment, CD45RO may be represented by Genebank ID No: 5788, which is provided herein as SEQ ID No: 19, as follows:

[SEQ ID No: 19]
MTMYLWLKLLAFGFAFLDTEVFVTGQSPTPSPTDAYLNASETTTLSPSGSAVISTTTIATTPSKPTCDEK
YANITVDYLYNKETKLFTAKLNVNENVECGNNTCTNNEVHNLTECKNASVSISHNSCTAPDKTLILDVPP
GVEKFQLHDCTQVEKADTTICLKWKNIETFTCDTQNITYRFQCGNMIFDNKEIKLENLEPEHEYKCDSEI
LYNNHKFTNASKIIKTDFGSPGEPQIIFCRSEAAHQGVITWNPPQRSFHNFTLCYIKETEKDCLNLDKNL
IKYDLQNLKPYTKYVLSLHAYIIAKVQRNGSAAMCHFTTKSAPPSQVWNMTVSMTSDNSMHVKCRPPRDR
NGPHERYHLEVEAGNTLVRNESHKNCDFRVKDLQYSTDYTFKAYFHNGDYPGEPFILHHSTSYNSKALIA
FLAFLIIVTSIALLVVLYKIYDLHKKRSCNLDEQQELVERDDEKQLMNVEPIHADILLETYKRKIADEGR
LFLAEFQSIPRVFSKFPIKEARKPFNQNKNRYVDILPYDYNRVELSEINGDAGSNYINASYIDGFKEPRK
YIAAQGPRDETVDDFWRMIWEQKATVIVMVTRCEEGNRNKCAEYWPSMEEGTRAFGDVVVKINQHKRCPD
YIIQKLNIVNKKEKATGREVTHIQFTSWPDHGVPEDPHLLLKLRRRVNAFSNFFSGPIVVHCSAGVGRTG
TYIGIDAMLEGLEAENKVDVYGYVVKLRRQRCLMVQVEAQYILIHQALVEYNQFGETEVNLSELHPYLHN
MKKRDPPSEPSPLEAEFQRLPSYRSWRTQHIGNQEENKSKNRNSNVIPYDYNRVPLKHELEMSKESEHDS
DESSDDDSDSEEPSKYINASFIMSYWKPEVMIAAQGPLKETIGDFWQMIFQRKVKVIVMLTELKHGDQEI
CAQYWGEGKQTYGDIEVDLKDTDKSSTYTLRVFELRHSKRKDSRTVYQYQYTNWSVEQLPAEPKELISMI
QVVKQKLPQKNSSEGNKHHKSTPLLIHCRDGSQQTGIFCALLNLLESAETEEVVDIFQVVKALRKARPGM
VSTFEQYQFLYDVIASTYPAQNGQVKKNNHQEDKIEFDNEVDKVKQDANCVNPLGAPEKLPEAKEQAEGS
EPTSGTEGPEHSVNGPASPALNQGS

Thus, preferably CD45RO comprises or consists of a sequence as substantially set out in SEQ ID No: 19, or a fragment or variant thereof.

In one embodiment, CCR7 may be represented by Genebank ID No: 1236, which is provided herein as SEQ ID No: 20, as follows:

[SEQ ID No: 20]
MDLGKPMKSVLVVALLVIFQVCLCQDEVTDDYIGDNTTVDYTLFESLCSKKDVRNFKAWFLPIMYSIICF
VGLLGNGLVVLTYIYFKRLKTMTDTYLLNLAVADILFLLTLPFWAYSAAKSWVFGVHFCKLIFAIYKMSF
FSGMLLLLCISIDRYVAIVQAVSAHRHRARVLLISKLSCVGIWILATVLSIPELLYSDLQRSSSEQAMRC
SLITEHVEAFITIQVAQMVIGFLVPLLAMSFCYLVIIRTLLQARNFERNKAIKVIIAVVVVFIVFQLPYN
GVVLAQTVANFNITSSTCELSKQLNIAYDVTYSLACVRCCVNPFLYAFIGVKFRNDLFKLFKDLGCLSQE
QLRQWSSCRHIRRSSMSVEAETTTTFSP

Thus, preferably CCR7 comprises or consists of a sequence as substantially set out in SEQ ID No: 20, or a fragment or variant thereof.

In one embodiment, CD27 may be represented by Genebank ID No: 939, which is provided herein as SEQ ID No: 21, as follows:

[SEQ ID No: 21]
MARPHPWWLCVLGTLVGLSATPAPKSCPERHYWAQGKLCCQMCEPGTFLVKDCDQHRKAAQCDPCIPGVS
FSPDHHTRPHCESCRHCNSGLLVRNCTITANAECACRNGWQCRDKECTECDPLPNPSLTARSSQALSPHP
QPTHLPYVSEMLEARTAGHMQTLADFRQLPARTLSTHWPPQRSLCSSDFIRILVIFSGMFLVFTLAGALF
LHQRRKYRSNKGESPVEPAEPCHYSCPREEEGSTIPIQEDYRKPEPACSP

Thus, preferably CD27 comprises or consists of a sequence as substantially set out in SEQ ID No: 21, or a fragment or variant thereof.

In one embodiment, CD62L may be represented by Genebank ID No: 6402, which is provided herein as SEQ ID No: 22, as follows:

[SEQ ID No: 22]
MGCRRTREGPSKAMIFPWKCQSTQRDLWNIFKLWGWTMLCCDFLAHHGTDCWTYHYSEKPMNWQRARRFC
RDNYTDLVAIQNKAEIEYLEKTLPFSRSYYWIGIRKIGGIWTWVGTNKSLTEEAENWGDGEPNNKKNKED
CVEIYIKRNKDAGKWNDDACHKLKAALCYTASCQPWSCSGHGECVEIINNYTCNCDVGYYGPQCQFVIQC
EPLEAPELGTMDCTHPLGNFSFSSQCAFSCSEGTNLTGIEETTCGPFGNWSSPEPTCQVIQCEPLSAPDL
GIMNCSHPLASFSFTSACTFICSEGTELIGKKKTICESSGIWSNPSPICQKLDKSFSMIKEGDYNPLFIP
VAVMVTAFSGLAFIIWLARRLKKGKKSKRSMNDPY

Thus, preferably CD62L comprises or consists of a sequence as substantially set out in SEQ ID No: 22, or a fragment or variant thereof.

Preferably, the lymphocyte has been obtained from tissue of a human or non-human animal. Preferably, the non-human animal is a mammal. The non-human animal may be a rodent, dog, horse or pig. The rodent may be a rat or a mouse. Preferably, the lymphocyte has been obtained from tissue of a human. The tissue may be selected from the group consisting of: blood, spleen, lymph node, lung, gastrointestinal tract, skin, prostate mammary gland tissue, liver, bone marrow and pancreas. Preferably, the tissue is blood or bone marrow.

The method may comprise obtaining the lymphocyte from a tissue obtained from a human or non-human animal. The lymphocyte may be obtained by any suitable method known in the art. Such methods include buffy coats or density gradients, fluorescent activated cell sorting and/or magnetic activated cell sorting. These methods would be known by a person skilled in the art.

Preferably, the tissue-resident memory T cell (T_{RM}) produced by the method of the invention is a tissue-resident memory CD8+ T cell. Preferably, a plurality of tissue-resident memory T cells (T_{RM}) are produced using the method.

The tissue-resident memory CD8+ T cell may be characterised by expression of cluster of differentiation 8 (CD8), cluster of differentiation 69 (CD69), Zinc Finger Protein 683 (ZNF683/HOBIT), aryl hydrocarbon receptor (AhR) and/or cluster of differentiation 103 (CD103). The tissue-resident memory CD8+ (cytotoxic) T cell may be further characterised by the absence of killer cell lectin-like receptor subfamily G member (KLRG1) and/or Eomesodermin (Eomes).

Preferably, the tissue-resident memory CD8+ (cytotoxic) T cell may be characterised by the expression of CD8, CD69, Hobit, AhR and CD103. Preferably, the tissue-resident memory CD8+ (cytotoxic) T cell may be characterised by the expression of CD8, CD69, Hobit, AhR, CD103 and the absence of KLRG1 and Eomes expression.

In one embodiment, CD8 may be represented by Genebank ID No: 925, which is provided herein as SEQ ID No: 2, as follows:

[SEQ ID No: 2]
MALPVTALLLPLALLLHAARPSQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALSNSIMYFSHFVPVFLPAKPTTT
PAPRPPTPAPTIASQPLSLRPEACRPAAGGAVHTRGLDFACDIYIWAPLAGTCGVLLLSLVITLYCNHRN
RRRVCKCPRPVVKSGDKPSLSARYV

Thus, preferably CD8 comprises or consists of a sequence as substantially set out in SEQ ID No: 2, or a fragment or variant thereof.

In one embodiment, CD69 may be represented by Genebank ID No: 969, which is provided herein as SEQ ID No: 3, as follows:

[SEQ ID No: 3]
MSSENCFVAENSSLHPESGQENDATSPHFSTRHEGSFQVPVLCAVMNVVFITILIIALIALSVGQYNCPG
QYTFSMPSDSHVSSCSEDWVGYQRKCYFISTVKRSWTSAQNACSEHGATLAVIDSEKDMNFLKRYAGREE
HWVGLKKEPGHPWKWSNGKEFNNWFNVTGSDKCVFLKNTEVSSMECEKNLYWICNKPYK

Thus, preferably CD69 comprises or consists of a sequence as substantially set out in SEQ ID No: 3, or a fragment or variant thereof.

In one embodiment, CD103 may be represented by Genebank ID No: 3682, which is provided herein as SEQ ID No: 4, as follows:

[SEQ ID No: 4]
MWLFHTLLCIASLALLAAFNVDVARPWLTPKGGAPFVLSSLLHQDPSTNQTWLLVTSPRTKRTPGPLHRC
SLVQDEILCHPVEHVPIPKGRHRGVTVVRSHHGVLICIQVLVRRPHSLSSELTGTCSLLGPDLRPQAQAN
FFDLENLLDPDARVDTGDCYSNKEGGGEDDVNTARQRRALEKEEEEDKEEEEDEEEEEAGTEIAIILDGS
GSIDPPDFQRAKDFISNMMRNFYEKCFECNFALVQYGGVIQTEFDLRDSQDVMASLARVQNITQVGSVTK
TASAMQHVLDSIFTSSHGSRRKASKVMVVLTDGGIFEDPLNLTTVINSPKMQGVERFAIGVGEEFKSART
ARELNLIASDPDETHAFKVTNYMALDGLLSKLRYNIISMEGTVGDALHYQLAQIGFSAQILDERQVLLGA
VGAFDWSGGALLYDTRSRRGRFLNQTAAAAADAEAAQYSYLGYAVAVLHKTCSLSYIAGAPRYKHHGAVF
ELQKEGREASFLPVLEGEQMGSYFGSELCPVDIDMDGSTDFLLVAAPFYHVHGEEGRVYVYRLSEQDGSF
SLARILSGHPGFTNARFGFAMAAMGDLSQDKLTDVAIGAPLEGFGADDGASFGSVYIYNGHWDGLSASPS
QRIRASTVAPGLQYFGMSMAGGFDISGDGLADITVGTLGQAVVFRSRPVVRLKVSMAFTPSALPIGFNGV
VNVRLCFEISSVTTASESGLREALLNFTLDVDVGKQRRRLQCSDVRSCLGCLREWSSGSQLCEDLLLMPT
EGELCEEDCFSNASVKVSYQLQTPEGQTDHPQPILDRYTEPFAIFQLPYEKACKNKLFCVAELQLATTVS
QQELVVGLTKELTLNINLTNSGEDSYMTSMALNYPRNLQLKRMQKPPSPNIQCDDPQPVASVLIMNCRIG
HPVLKRSSAHVSVVWQLEENAFPNRTADITVTVTNSNERRSLANETHTLQFRHGFVAVLSKPSIMYVNTG
QGLSHHKEFLFHVHGENLFGAEYQLQICVPTKLRGLQVVAVKKLTRTQASTVCTWSQERACAYSSVQHVE
EWHSVSCVIASDKENVTVAAEISWDHSEELLKDVTELQILGEISFNKSLYEGLNAENHRTKITVVFLKDE
KYHSLPIIIKGSVGGLLVLIVILVILFKCGFFKRKYQQLNLESIRKAQLKSENLLEEEN

Thus, preferably CD103 comprises or consists of a sequence as substantially set out in SEQ ID No: 4, or a fragment or variant thereof.

In one embodiment, KLRG1 may be represented by Genebank ID No: 10219, which is provided herein as SEQ ID No: 5, as follows:

[SEQ ID No: 5]
MTDSVIYSMLELPTATQAQNDYGPQQKSSSSRPSCSCLVAIALGLLTAVLLSVLLYQWILCQGSNYSTCA
SCPSCPDRWMKYGNHCYYFSVEEKDWNSSLEFCLARDSHLLVITDNQEMSLLQVFLSEAFCWIGLRNNSG
WRWEDGSPLNFSRISSNSFVQTCGAINKNGLQASSCEVPLHWVCKKCPFADQALF

Thus, preferably KLRG1 comprises or consists of a sequence as substantially set out in SEQ ID No: 5, or a fragment or variant thereof.

In one embodiment, Eomes may be represented by Genebank ID No: 8320, which is provided herein as SEQ ID No: 6, as follows:

[SEQ ID No: 6]
MQLGEQLLVSSVNLPGAHFYPLESARGGSGGSAGHLPSAAPSPQKLDLDKASKKFSGSLSCEAVSGEPAA
ASAGAPAAMLSDTDAGDAFASAAAVAKPGPPDGRKGSPCGEEELPSAAAAAAAAAAAAAATARYSMDSLS
SERYYLQSPGPQGSELAAPCSLFPYQAAAGAPHGPVYPAPNGARYPYGSMLPPGGFPAAVCPPGRAQFGP
GAGAGSGAGGSSGGGGGPGTYQYSQGAPLYGPYPGAAAAGSCGGLGGLGVPGSGFRAHVYLCNRPLWLKF
HRHQTEMIITKQGRRMFPFLSFNINGLNPTAHYNVFVEVVLADPNHWRFQGGKWVTCGKADNNMQGNKMY
VHPESPNTGSHWMRQEISFGKLKLTNNKGANNNNTQMIVLQSLHKYQPRLHIVEVTEDGVEDLNEPSKTQ
TFTFSETQFIAVTAYQNTDITQLKIDHNPFAKGFRDNYDSSHQIVPGGRYGVQSFFPEPFVNTLPQARYY
NGERTVPQTNGLLSPQQSEEVANPPQRWLVTPVQQPGTNKLDISSYESEYTSSTLLPYGIKSLPLQTSHA
LGYYPDPTFPAMAGWGGRGSYQRKMAAGLPWTSRTSPTVFSEDQLSKEKVKEEIGSSWIETPPSIKSLDS
NDSGVYTSACKRRRLSPSNSSNENSPSIKCEDINAEEYSKDTSKGMGGYYAFYTTP

Thus, preferably Eomes comprises or consists of a sequence as substantially set out in SEQ ID No: 6, or a fragment or variant thereof.

In one embodiment, Hobit may be represented by Genebank ID No: 257101, which is provided herein as SEQ ID No: 9, as follows

[SEQ ID No: 9]
MKEESAAQLGCCHRPMALGGTGGSLSPSLDFQLFRGDQVFSACRPLPDMVDAHGPSCASWLCPLPLAPGR
SALLACLQDLDLNLCTPQPAPLGTDLQGLQEDALSMKHEPPGLQASSTDDKKFTVKYPQNKDKLGKQPER
AGEGAPCPAFSSHNSSSPPPLQNRKSPSPLAFCPCPPVNSISKELPFLLHAFYPGYPLLLPPPHLFTYGA
LPSDQCPHLLMLPQDPSYPTMAMPSLLMMVNELGHPSARWETLLPYPGAFQASGQALPSQARNPGAGAAP
TDSPGLERGGMASPAKRVPLSSQTGTAALPYPLKKKNGKILYECNICGKSFGQLSNLKVHLRVHSGERPF
QCALCQKSFTQLAHLQKHHLVHTGERPHKCSIPWVPGRNHWKSFQAWREREVCHKRFSSSSNLKTHLRLH
SGARPFQCSVCRSRFTQHIHLKLHHRLHAPQPCGLVHTQLPLASLACLAQWHQGALDLMAVASEKHMGYD
IDEVKVSSTSQGKARAVSLSSAGTPLVMGQDQNN

Thus, preferably Hobit comprises or consists of a sequence as substantially set out in SEQ ID No: 9, or a fragment or variant thereof.

In one embodiment, Ahr may be represented by Genebank ID No: 196, which is provided herein as SEQ ID No: 10, as follows

[SEQ ID No: 10]
MNSSSANITYASRKRRKPVQKTVKPIPAEGIKSNPSKRHRDRLNTELDRLASLLPFPQDVINKLDKLSVL
RLSVSYLRAKSFFDVALKSSPTERNGGQDNCRAANFREGLNLQEGEFLLQALNGFVLVVTTDALVFYASS
TIQDYLGFQQSDVIHQSVYELIHTEDRAEFQRQLHWALNPSQCTESGQGIEEATGLPQTVVCYNPDQIPP
ENSPLMERCFICRLRCLLDNSSGFLAMNFQGKLKYLHGQKKKGKDGSILPPQLALFAIATPLQPPSILEI
RTKNFIFRTKHKLDFTPIGCDAKGRIVLGYTEAELCTRGSGYQFIHAADMLYCAESHIRMIKTGESGMIV
FRLLTKNNRWTWVQSNARLLYKNGRPDYIIVTQRPLTDEEGTEHLRKRNTKLPFMFTTGEAVLYEATNPF
PAIMDPLPLRTKNGTSGKDSATTSTLSKDSLNPSSLLAAMMQQDESIYLYPASSTSSTAPFENNFFNESM
NECRNWQDNTAPMGNDTILKHEQIDQPQDVNSFAGGHPGLFQDSKNSDLYSIMKNLGIDFEDIRHMQNEK
FFRNDFSGEVDFRDIDLTDEILTYVQDSLSKSPFIPSDYQQQQSLALNSSCMVQEHLHLEQQQQHHQKQV
VVEPQQQLCQKMKHMQVNGMFENWNSNQFVPFNCPQQDPQQYNVFTDLHGISQEFPYKSEMDSMPYTQNF
ISCNQPVLPQHSKCTELDYPMGSFEPSPYPTTSSLEDFVTCLQLPENQKHGLNPQSAIITPQTCYAGAVS
MYQCQPEPQHTHVGQMQYNPVLPGQQAFLNKFQNGVLNETYPAELNNINNTQTTTHLQPLHHPSEARPFP
DLTSSGFL

Thus, preferably Ahr comprises or consists of a sequence as substantially set out in SEQ ID No: 10, or a fragment or variant thereof.

Preferably, the method comprises culturing the lymphocyte in the presence of interleukin 2, 4, 7, 12, 15 and/or 21 (IL-2, IL-4, IL-7, IL-12 IL-15 and/or IL-21).

The interleukin is preferably mammalian, and most preferably a human interleukin.

Preferably, the method comprises culturing the lymphocyte in the presence of interleukin 7 (IL-7).

Preferably, the IL-7 is mammalian. Most preferably, the IL-7 is human IL-7. In one embodiment, IL-7 may be represented by Genebank ID No: 3574 which is provided herein as SEQ ID No: 28, as follows:

[SEQ ID No: 28]
MFHVSFRYIFGLPPLILVLLPVASSDCDIEGKDGKQYESVLMVSIDQLLDSMKEIGSNCLNNEFNFFKRH
ICDANKEGMFLFRAARKLRQFLKMNSTGDFDLHLLKVSEGTTILLNCTGQVKGRKPAALGEAQPTKSLEE
NKSLKEQKKLNDLCFLKRLLQEIKTCWNKILMGTKEH

Thus, preferably IL-7 comprises or consists of a sequence as substantially set out in SEQ ID No: 28, or a fragment or variant thereof.

Preferably, IL-7 may be present at a concentration of between 0.1 ng/ml and 200 ng/ml. More preferably, IL-7 may be present at a concentration of between 2 ng/ml and 100 ng/ml. Most preferably, IL-7 may be present at a concentration of between 10 ng/ml and 50 ng/ml.

Preferably, the method further comprises culturing the lymphocyte in the presence of interleukin 15 (IL-15).

Preferably, the IL-15 is mammalian. Most preferably, the IL-15 is human IL-15. In one embodiment, IL-15 may be represented by Genebank ID No: 3600, which is provided herein as SEQ ID No: 7, as follows:

[SEQ ID No: 7]
MRISKPHLRSISIQCYLCLLLNSHFLTEAGIHVFILGCFSAGLPKTEANWVNVISDLKKIEDLIQSMHID
ATLYTESDVHPSCKVTAMKCFLLELQVISLESGDASIHDTVENLIILANNSLSSNGNVTESGCKECEELE
EKNIKEFLQSFVHIVQMFINTS

Thus, preferably IL-15 comprises or consists of a sequence as substantially set out in SEQ ID No: 7, or a fragment or variant thereof.

Preferably, IL-15 may be present at a concentration of between 1 ng/ml and 100 ng/ml. More preferably, the IL-15 may be present at a concentration of between 5 ng/ml and 50 ng/ml. Most preferably, IL-15 may be present at a concentration of between 10 ng/ml and 25 ng/ml.

Preferably, the method further comprises culturing in the presence of interleukin 33 (IL-33). Preferably, the IL-33 is mammalian. Most preferably, the IL-33 is human IL-33. In one embodiment, IL-33 may be represented by Genebank ID No: 90865, which is provided herein as SEQ ID No: 8, as follows:

[SEQ ID No: 8]
MKPKMKYSTNKISTAKWKNTASKALCFKLGKSQQKAKEVCPMYFMKLRSGLMIKKEACYFRRETTKRPSL
KTGRKHKRHLVLAACQQQSTVECFAFGISGVQKYTRALHDSSITGISPITEYLASLSTYNDQSITFALED
ESYEIYVEDLKKDEKKDKVLLSYYESQHPSNESGDGVDGKMLMVTLSPTKDFWLHANNKEHSVELHKCEK
PLPDQAFFVLHNMHSNCVSFECKTDPGVFIGVKDNHLALIKVDSSENLCTENILFKLSET

Thus, preferably IL-33 comprises or consists of a sequence as substantially set out in SEQ ID No: 8, or a fragment or variant thereof.

Preferably, IL-33 may be present at a concentration of between 0.5 ng/ml and 100 ng/ml. More preferably, the IL-33 may be present at a concentration of between 2 ng/ml and 50 ng/ml. Most preferably, IL-33 may be present at a concentration of between 10 ng/ml and 25 ng/ml.

Preferably, the method comprises culturing the lymphocyte in the presence of interleukin 2 (IL-2).

Thus, in one embodiment, there is provided a method for producing a tissue-resident memory T cell (T_{RM}), the method comprising culturing a naive CD8+ T lymphocyte in the presence of TGFβ.

In another embodiment, there is provided a method for producing a tissue-resident memory T cell, the method comprising culturing a naive CD8+ T lymphocyte in the presence of TGFβ, IL-15 and IL-33.

Thus, in one embodiment, there is provided a method for producing a tissue-resident memory CD8+ (cytotoxic) T cell, the method comprising culturing a naive CD8+ T lymphocyte in the presence of TGFβ, IL-15 and IL-33.

Preferably, the method further comprises culturing in the presence of at least one interleukin 1 family member, for example IL-1α, IL-1β and/or IL-18.

Preferably, the interleukin 1 family member is mammalian. Most preferably, the interleukin 1 family member is human.

In one embodiment, IL-1α may be represented by Genebank ID No: 3552, which is provided herein as SEQ ID No: 11, as follows:

[SEQ ID No: 11]
MAKVPDMFEDLKNCYSENEEDSSSIDHLSLNQKSFYHVSYGPLHEGCMDQSVSLSISETSKTSKLTFKES
MVVVATNGKVLKKRRLSLSQSITDDDLEAIANDSEEEIIKPRSAPFSFLSNVKYNFMRIIKYEFILNDAL
NQSIIRANDQYLTAAALHNLDEAVKFDMGAYKSSKDDAKITVILRISKTQLYVTAQDEDQPVLLKEMPEI
PKTITGSETNLLFFWETHGTKNYFTSVAHPNLFIATKQDYWVCLAGGPPSITDFQILENQA

Thus, preferably IL-1α comprises or consists of a sequence as substantially set out in SEQ ID No: 11, or a fragment or variant thereof.

Preferably, IL-1α may be present at a concentration of between 0.1 ng/ml and 100 ng/ml. More preferably, IL-1α may be present at a concentration of between 1 ng/ml and 50 ng/ml. Most preferably, IL-1α may be present at a concentration of between 5 ng/ml and 20 ng/ml.

In one embodiment, IL-1β may be represented by Genebank ID No: 3553, which is provided herein as SEQ ID No: 12, as follows:

[SEQ ID No: 12]
MAEVPELASEMMAYYSGNEDDLFFEADGPKQMKCSFQDLDLCPLDGGIQLRISDHHYSKGFRQAASVVVA
MDKLRKMLVPCPQTFQENDLSTFFPFIFEEEPIFFDTWDNEAYVHDAPVRSLNCTLRDSQQKSLVMSGPY
ELKALHLQGQDMEQQVVFSMSFVQGEESNDKIPVALGLKEKNLYLSCVLKDDKPTLQLESVDPKNYPKKK
MEKRFVFNKIEINNKLEFESAQFPNWYISTSQAENMPVFLGGTKGGQDITDFTMQFVSS

Thus, preferably IL-1β comprises or consists of a sequence as substantially set out in SEQ ID No: 12, or a fragment or variant thereof.

Preferably, IL-1β may be present at a concentration of between 0.1 ng/ml and 100 ng/ml. More preferably, IL-1β may be present at a concentration of between 1 ng/ml and 50 ng/ml. Most preferably, IL-1β may be present at a concentration of between 5 ng/ml and 20 ng/ml.

In one embodiment, IL-18 may be represented by Genebank ID No: 3606, which is provided herein as SEQ ID No: 13, as follows:

[SEQ ID No: 13]
MAAEPVEDNCINFVAMKFIDNTLYFIAEDDENLESDYFGKLESKLSVIRNLNDQVLFIDQGNRPLFEDMT
DSDCRDNAPRTIFIISMYKDSQPRGMAVTISVKCEKISTLSCENKIISFKEMNPPDNIKDTKSDIIFFQR
SVPGHDNKMQFESSSYEGYFLACEKERDLFKLILKKEDELGDRSIMFTVQNED

Thus, preferably IL-18 comprises or consists of a sequence as substantially set out in SEQ ID No: 13, or a fragment or variant thereof.

Preferably, IL-18 may be present at a concentration of between 0.1 ng/ml and 100 ng/ml. More preferably, IL-18 may be present at a concentration of between 1 ng/ml and 50 ng/ml. Most preferably, IL-18 may be present at a concentration of between 5 ng/ml and 20 ng/ml.

The lymphocytes may be cultured in a culture media comprising at least one aryl hydrocarbon receptor (AhR) ligand. The AhR ligand may be an agonist or an antagonist. Preferably, the AhR ligand is an agonist. Preferably, the AhR ligand is an antagonist.

The AhR ligand may be selected from a group consisting of a halogenated aromatic hydrocarbon, a polycyclic aromatic hydrocarbon, a dietary derived aryl hydrocarbon, a heme metabolite, an indigoid, StemRegenin 1 and a tryptophan metabolite.

The halogenated aromatic hydrocarbon may be tetrachlorodibenzo-p-dioxin (TCDD). The polycyclic aromatic hydrocarbon may be 3-methylcholanthrene. The tryptophan metabolite may be 6-formylindolo [3,2-b] carbazole (FICZ). The dietary derived aryl hydrocarbon may be a flavone and/or indole-derivative. The indole-derivative may be Indole-3-Carbinol (I3C) and/or its product Diindolylmethane (DIM).

The lymphocyte may be cultured in a culture media comprising at least one lipid. Preferably, the lipid is cholesterol and/or medium chain fatty acids (MCFAs). The MCFA may be oleic acid.

The lymphocyte may be further cultured with an antigen. The specific type of antigen will depend on the therapeutic application for which the T_{RM} cells are to be used. For example, the lymphocyte may be cultured with a tumour antigen.

Preferably, in some embodiments, the method comprises culturing the lymphocyte in the presence of a regulatory T cell or a type 1 regulatory T cell.

Hence, in another aspect of the invention, there is provided a method for producing a tissue-resident memory T cell (T_{RM}), the method comprising culturing a lymphocyte in the presence of transforming growth factor beta (TGFβ) and/or co-culturing the lymphocyte with a type 1 regulatory T cell.

The skilled person would understand that a “regulatory T cell” is a T cell participating in peripheral immunity as a subset of CD4+ T cells. Preferably, regulatory T cells are characterised by expression of the transcription factor, forkhead box P3 (Foxp3). In other embodiments, the method does not comprise culturing the lymphocyte in the presence of a regulatory T cell.

The skilled person would understand that a “type 1 regulatory T cell” is a class of regulatory T cells participating in peripheral immunity as a subset of CD4+ T cells. Preferably, the type 1 regulatory T cell is characterised by expression of the transcription factors, forkhead box P3 (Foxp3), T-box transcription factor 21 (Tbet), and/or surface molecule C-X-C motif chemokine receptor 3 (CXCR3). In other embodiments, the method does not comprise culturing the lymphocyte in the presence of a type 1 regulatory T cell.

In one embodiment, Foxp3 may be represented by Genebank ID No: 50943, which is provided herein as SEQ ID No: 23, as follows:

[SEQ ID No: 23]
MPNPRPGKPSAPSLALGPSPGASPSWRAAPKASDLLGARGPGGTFQGRDLRGGAHASSSSLNPMPPSQLQ
LPTLPLVMVAPSGARLGPLPHLQALLQDRPHFMHQLSTVDAHARTPVLQVHPLESPAMISLTPPTTATGV
FSLKARPGLPPGINVASLEWVSREPALLCTFPNPSAPRKDSTLSAVPQSSYPLLANGVCKWPGCEKVFEE
PEDFLKHCQADHLLDEKGRAQCLLQREMVQSLEQQLVLEKEKLSAMQAHLAGKMALTKASSVASSDKGSC
CIVAAGSQGPVVPAWSGPREAPDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVESEKGAVWTVDELEFRKKRSQR
PSRCSNPTPGP

Thus, preferably Foxp3 comprises or consists of a sequence as substantially set out in SEQ ID No: 23, or a fragment or variant thereof.

In one embodiment, Tbet may be represented by Genebank ID No: 30009, which is provided herein as SEQ ID No: 24, as follows:

[SEQ ID No: 24]
MGIVEPGCGDMLTGTEPMPGSDEGRAPGADPQHRYFYPEPGAQDADERRGGGSLGSPYPGGALVPAPPSR
FLGAYAYPPRPQAAGFPGAGESFPPPADAEGYQPGEGYAAPDPRAGLYPGPREDYALPAGLEVSGKLRVA
LNNHLLWSKFNQHQTEMIITKQGRRMFPFLSFTVAGLEPTSHYRMFVDVVLVDQHHWRYQSGKWVQCGKA
EGSMPGNRLYVHPDSPNTGAHWMRQEVSFGKLKLTNNKGASNNVTQMIVLQSLHKYQPRLHIVEVNDGEP
EAACNASNTHIFTFQETQFIAVTAYQNAEITQLKIDNNPFAKGFRENFESMYTSVDTSIPSPPGPNCQFL
GGDHYSPLLPNQYPVPSRFYPDLPGQAKDVVPQAYWLGAPRDHSYEAEFRAVSMKPAFLPSAPGPTMSYY
RGQEVLAPGAGWPVAPQYPPKMGPASWFRPMRTLPMEPGPGGSEGRGPEDQGPPLVWTEIAPIRPESSDS
GLGEGDSKRRRVSPYPSSGDSSSPAGAPSPFDKEAEGQFYNYFPN

Thus, preferably Tbet comprises or consists of a sequence as substantially set out in SEQ ID No: 24, or a fragment or variant thereof.

In one embodiment, CXCR3 may be represented by Genebank ID No: 2833, which is provided herein as SEQ ID No: 25, as follows:

[SEQ ID No: 25]
MELRKYGPGRLAGTVIGGAAQSKSQTKSDSITKEFLPGLYTAPSSPFPPSQVSDHQVLNDAEVAALLENF
SSSYDYGENESDSCCTSPPCPQDFSLNFDRAFLPALYSLLFLLGLLGNGAVAAVLLSRRTALSSTDTFLL
HLAVADTLLVLTLPLWAVDAAVQWVFGSGLCKVAGALFNINFYAGALLLACISFDRYLNIVHATQLYRRG
PPARVTLTCLAVWGLCLLFALPDFIFLSAHHDERLNATHCQYNFPQVGRTALRVLQLVAGFLLPLLVMAY
CYAHILAVLLVSRGQRRLRAMRLVVVVVVAFALCWTPYHLVVLVDILMDLGALARNCGRESRVDVAKSVT
SGLGYMHCCLNPLLYAFVGVKFRERMWMLLLRLGCPNQRGLQRQPSSSRRDSSWSETSEASYSGL

Thus, preferably CXCR3 comprises or consists of a sequence as substantially set out in SEQ ID No: 25, or a fragment or variant thereof.

Preferably, the regulatory T cell (preferably type 1 regulatory T cell) expresses integrin alpha V beta 8 (αvβ8). The skilled person would understand that αvβ8 is a dimer of integrin subunit (Itgβ8) and integrin subunit alpha V (Itgav). Thus, preferably, the regulatory T cell (preferably type 1 regulatory T cell) expresses Itgβ8 and Itgva.

In one embodiment, Itgβ8 may be represented by Genebank ID No: 3696, which is provided herein as SEQ ID No: 26, as follows:

[SEQ ID No: 26]
MCGSALAFFTAAFVCLQNDRRGPASFLWAAWVFSLVLGLGQGEDNRCASSNAASCARCLALGPECGWCVQ
EDFISGGSRSERCDIVSNLISKGCSVDSIEYPSVHVIIPTENEINTQVTPGEVSIQLRPGAEANFMLKVH
PLKKYPVDLYYLVDVSASMHNNIEKLNSVGNDLSRKMAFFSRDFRLGFGSYVDKTVSPYISIHPERIHNQ
CSDYNLDCMPPHGYIHVLSLTENITEFEKAVHRQKISGNIDTPEGGFDAMLQAAVCESHIGWRKEAKRLL
LVMTDQTSHLALDSKLAGIVVPNDGNCHLKNNVYVKSTTMEHPSLGQLSEKLIDNNINVIFAVQGKQFHW
YKDLLPLLPGTIAGEIESKAANLNNLVVEAYQKLISEVKVQVENQVQGIYFNITAICPDGSRKPGMEGCR
NVTSNDEVLFNVTVTMKKCDVTGGKNYAIIKPIGFNETAKIHIHRNCSCQCEDNRGPKGKCVDETFLDSK
CFQCDENKCHFDEDQFSSESCKSHKDQPVCSGRGVCVCGKCSCHKIKLGKVYGKYCEKDDFSCPYHHGNL
CAGHGECEAGRCQCFSGWEGDRCQCPSAAAQHCVNSKGQVCSGRGTCVCGRCECTDPRSIGRFCEHCPTC
YTACKENWNCMQCLHPHNLSQAILDQCKTSCALMEQQHYVDQTSECFSSPSYLRIFFIIFIVTFLIGLLK
VLIIRQVILQWNSNKIKSSSDYRVSASKKDKLILQSVCTRAVTYRREKPEEIKMDISKLNAHETFRCNF

Thus, preferably Itgβ8 comprises or consists of a sequence as substantially set out in SEQ ID No: 26, or a fragment or variant thereof.

In one embodiment, Itgav may be represented by Genebank ID No: 3685, which is provided herein as SEQ ID No: 27, as follows:

[SEQ ID No: 27]
MAFPPRRRLRLGPRGLPLLLSGLLLPLCRAFNLDVDSPAEYSGPEGSYFGFAVDFFVPSASSRMFLLVGA
PKANTTQPGIVEGGQVLKCDWSSTRRCQPIEFDATGNRDYAKDDPLEFKSHQWFGASVRSKQDKILACAP
LYHWRTEMKQEREPVGTCFLQDGTKTVEYAPCRSQDIDADGQGFCQGGFSIDFTKADRVLLGGPGSFYWQ
GQLISDQVAEIVSKYDPNVYSIKYNNQLATRTAQAIFDDSYLGYSVAVGDFNGDGIDDFVSGVPRAARTL
GMVYIYDGKNMSSLYNFTGEQMAAYFGFSVAATDINGDDYADVFIGAPLFMDRGSDGKLQEVGQVSVSLQ
RASGDFQTTKLNGFEVFARFGSAIAPLGDLDQDGFNDIAIAAPYGGEDKKGIVYIFNGRSTGLNAVPSQI
LEGQWAARSMPPSFGYSMKGATDIDKNGYPDLIVGAFGVDRAILYRARPVITVNAGLEVYPSILNQDNKT
CSLPGTALKVSCFNVRFCLKADGKGVLPRKLNFQVELLLDKLKQKGAIRRALFLYSRSPSHSKNMTISRG
GLMQCEELIAYLRDESEFRDKLTPITIFMEYRLDYRTAADTTGLQPILNQFTPANISRQAHILLDCGEDN
VCKPKLEVSVDSDQKKIYIGDDNPLTLIVKAQNQGEGAYEAELIVSIPLQADFIGVVRNNEALARLSCAF
KTENQTRQVVCDLGNPMKAGTQLLAGLRFSVHQQSEMDTSVKFDLQIQSSNLFDKVSPVVSHKVDLAVLA
AVEIRGVSSPDHVFLPIPNWEHKENPETEEDVGPVVQHIYELRNNGPSSFSKAMLHLQWPYKYNNNTLLY
ILHYDIDGPMNCTSDMEINPLRIKISSLQTTEKNDTVAGQGERDHLITKRDLALSEGDIHTLGCGVAQCL
KIVCQVGRLDRGKSAILYVKSLLWTETFMNKENQNHSYSLKSSASFNVIEFPYKNLPIEDITNSTLVTTN
VTWGIQPAPMPVPVWVIILAVLAGLLLLAVLVFVMYRMGFFKRVRPPQEEQEREQLQPHENGEGNSET

Thus, preferably Itgav comprises or consists of a sequence as substantially set out in SEQ ID No: 27, or a fragment or variant thereof.

In one embodiment, the type 1 regulatory T cell (preferably type 1 regulatory T cell) may be activated, preferably by an anti-CD3 molecule, such as an anti-CD3 antibody, and/or IL-2.

Expression of αvβ8 may be enhanced in the regulatory T cell (preferably type 1 regulatory T cell) with amphigerulin. Thus, the method may further comprise contacting the regulatory T cell (preferably type 1 regulatory T cell) with amphigerulin.

The method may further comprise culturing the lymphocyte with a dendritic cell.

Preferably, the lymphocyte is cultured with between 100E+03 cells/cm² and 2000E+03 cells/cm². More preferably, the lymphocyte is cultured with between 250E+03 cells/cm² and 1000E+03 cells/cm². Most preferably, the lymphocyte is cultured with between 250E+03 cells/cm² and 1000E+03 cells/cm².

In one embodiment, the method further comprises purifying the T_{RM} cells from the culture.

The skilled person would understand that any factor, such as cytokines, described herein may be mammalian. The mammal may be a rodent, dog, horse or pig. The rodent may be a rat or a mouse. However, preferably, the factors described herein are human.

In a second aspect, there is provided a method of expanding a population of tissue-resident memory T cells (T_{RM}), the method comprising culturing a population of tissue resident memory T cells as defined in the first aspect.

The tissue-resident memory T cells may be as defined in the first aspect.

The method of the first or second aspect may further comprise culturing the tissue resident memory T cells in the presence of IL-2, IL-4, IL-7, IL-12, IL-15 and/or IL-21.

Preferably the IL-2 is mammalian. Most preferably, the IL-2 is human IL-2. In one embodiment, IL-2 may be represented by Genebank ID No: 3558, which is provided herein as SEQ ID No: 17, as follows:

[SEQ ID No: 17]
MYRMQLLSCIALSLALVTNSAPTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSETTFMCEYADETATIVEFLNR
WITFCQSIISTLT

Thus, preferably IL-2 comprises or consists of a sequence as substantially set out in SEQ ID No: 17, or a fragment or variant thereof.

Preferably, IL-2 may be present at a concentration of between 0.1 ng/ml and 200 ng/ml. More preferably, IL-2 may be present at a concentration of between 2 ng/ml and 100 ng/ml. Most preferably, IL-2 may be present at a concentration of between 10 ng/ml and 50 ng/ml.

Preferably, the IL-21 is mammalian. Most preferably, the IL-21 is human IL-21. In one embodiment, IL-21 may be represented by Genebank ID No: 59067, which is provided herein as SEQ ID No: 15, as follows:

[SEQ ID No: 15]
MRSSPGNMERIVICLMVIFLGTLVHKSSSQGQDRHMIRMRQLIDIVDQLKNYVNDLVPEFLPAPEDVETN
CEWSAFSCFQKAQLKSANTGNNERIINVSIKKLKRKPPSTNAGRRQKHRLTCPSCDSYEKKPPKEFLERF
KSLLQKMIHQHLSSRTHGSEDS

Thus, preferably IL-21 comprises or consists of a sequence as substantially set out in SEQ ID No: 15, or a fragment or variant thereof.

Preferably, IL-21 may be present at a concentration of between 0.1 ng/ml and 200 ng/ml. More preferably, IL-21 may be present at a concentration of between 5 ng/ml and 100 ng/ml. Most preferably, IL-21 may be present at a concentration of between 10 ng/ml and 50 ng/ml.

In a third aspect, there is provided a tissue-resident memory T cell obtained, or obtainable, by the method according to the first aspect.

The tissue resident memory T cells produced by the methods of the invention are particularly useful in therapeutic applications.

T cells have been shown to be a powerful tool to eradicate tumours. The presence of T cells, and in particular tissue resident memory T cells within tumour tissue is known to correlate with a positive cancer prognosis⁹⁴. Several studies have particularly associated the presence of T_{RM} cells, identified by their expression of CD103, in solid tumours with a high survival rate and an overall positive prognosis, even in advanced stages of cancer^{95,96}. Furthermore, T_{RM} cells have been shown to significantly improve the survival rate of cancer patients in combination therapy with other known immunotherapeutics⁹⁷. However, the number of naturally occurring T_{RM} cells is usually low compared to other types of T cells, to observe a significant therapeutic effect. As such, generating in vitro T cells for anti-tumour therapy with attributes of T_{RM} cells and their migratory and tissue homing attributes, such as expression of CD103, CD69 and CTLA-4, which allow the T cells to penetrate tumours would be highly valuable as a mono or a combination therapy.

Thus, preferably the T_{RM} cell of the invention expresses CD103. Preferably, the T_{RM} cell of the invention expresses CD69. Preferably, the T_{RM} cell of the invention expresses CTLA-4.

Accordingly, in a fourth aspect of the invention, there is provided the tissue-resident memory T cell according to the third aspect, optionally an expanded population thereof, for use in therapy.

In a fifth aspect of the invention, there is provided a tissue resident memory T cell according to the third aspect, optionally an expanded population thereof, for use in T cell therapy.

It will be appreciated that an expanded population of tissue-resident memory T cells (T_{RM}) would be especially useful in therapy, especially T cell therapies.

The T cell therapy may be CAR-T cell therapy. The T-cell therapy may be innate-like T cell therapy such as gamma delta T cell, mucosal associated invariant T cell or natural killer T cells-based therapy.

In a sixth aspect of the invention, there is provided a tissue resident memory T cell according to the third aspect, optionally an expanded population thereof, for use in the prevention, treatment or amelioration of cancer or an infection.

In a seventh aspect of the invention, there is provided a method of treating cancer or an infection in a subject, the method comprising administering, or having administered, to a subject in need of such treatment, a therapeutically effective amount of the tissue resident memory T cell according to the third aspect, optionally an expanded population thereof.

T_{RM} cells may be generated by in vitro culture of previously activated T cells in the presence of antigen presenting cells, interleukin (IL)-15 and TGFβ. Addition of IL-2 or particularly IL-7 preferably enhances the T_{RM} cell migration properties, such as the expression of cluster of differentiation (CD) 69, CD103 and cytotoxic T-lymphocyte-associated protein 4 (CTLA-4), and cell recovery from peripheral organs upon adoptive transfer in a mouse model.

It will be appreciated that the tissue resident memory T cells produced according to the invention may be used in a monotherapy (i.e. the sole use of (i) a tissue resident memory T cell or (ii) a therapeutic composition comprising issue resident memory T cells). Alternatively, tissue resident memory T cells according to the invention may be used as an adjunct to, or in combination with, known therapies for treating, ameliorating, or preventing disease, for example cancer.

The tissue resident memory T cells according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

The tissue resident memory T cells of the invention may be used in a number of ways. For instance, oral administration may be required, in which case the agents may be contained within a composition that may, for example, be ingested orally in the form of a tablet, capsule or liquid. Antibiotic compositions and formulations of the invention may be administered by inhalation (e.g., intranasally). Compositions may also be formulated for topical use. For instance, creams or ointments may be applied to the skin.

Tissue resident memory T cells compositions and formulations according to the invention may also be incorporated within a slow- or delayed-release device. Such devices may, for example, be inserted on or under the skin at a specific tissue location, and the medicament may be released over hours, days, weeks or even months. The device may be located at least adjacent to the treatment site. Such devices may be particularly advantageous when long-term treatment with agents used according to the invention is required and which would normally require frequent administration (e.g. at least daily administration).

In a preferred embodiment, the tissue resident memory T cells according to the invention may be administered to a subject by injection into the blood stream or directly into a site requiring treatment. Injections may be intravenous (bolus or infusion) or subcutaneous (bolus or infusion), or intradermal (bolus or infusion), or intramuscular. Preferably the tissue resident memory T cells of the invention are administered via peripheral blood. Preferably, the tissue resident memory T cells of the invention are administered intravenous.

It will be appreciated that the amount of the tissue resident memory T cells that are required is determined by its biological activity and bioavailability, which in turn depends on the mode of administration, the physiochemical properties of the Tissue resident memory T cells, and whether they are being used as a monotherapy or in a combined therapy. The frequency of administration will also be influenced by the half-life of the tissue resident memory T cells within the subject being treated. Optimal dosages to be administered may be determined by those skilled in the art, and will vary with the particular compositions and formulations in use, the strength of the pharmaceutical composition, the mode of administration, and the advancement of the specific disease to be treated. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of the T_{RM} cells or formulation according to the invention may be used, depending upon which composition or formulation is used. More preferably, the daily dose is between 0.01 μg/kg of body weight and 1 mg/kg of body weight, more preferably between 0.1 μg/kg and 100 μg/kg body weight, and most preferably between approximately 0.1 μg/kg and 10 μg/kg body weight. Generally, a daily dose of between 10\hat{ }5 T_{RM} cells and 10\hat{ }7 T_{RM} cells of the invention may be used. Preferably, a daily dose of between 10\hat{ }5 T_{RM} cells and 10\hat{ }6 T_{RM} cells of the invention may be used.

The composition or formulation may be administered before, during or after onset of the disease to be treated. Daily doses may be given as a single administration (e.g., a single daily injection). Alternatively, the tissue resident memory T cells may require administration twice or more times during a day. As an example, the tissue resident memory T cells may be administered as two (or more depending upon the severity of the disease being treated) daily doses of between 10\hat{ }5 T_{RM} cells and 10\hat{ }7 T_{RM} cells (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of concentration according to the invention to a patient without the need to administer repeated doses.

Known procedures, such as those conventionally employed by the pharmaceutical industry (e.g., in vivo experimentation, clinical trials, etc.), may be used to form specific formulations according to the invention and precise therapeutic regimes (such as daily doses of the tissue resident memory T cells and the frequency of administration).

There is provided, in an eighth aspect, a pharmaceutical composition comprising a tissue-resident memory T cell according to the third aspect, optionally an expanded population thereof, and a pharmaceutically acceptable excipient.

The invention also provides in an ninth aspect, a process for making the pharmaceutical composition according to the eighth aspect, the process comprising combining a therapeutically effective amount of a tissue resident memory T cell according to the third aspect, optionally an expanded population thereof, with a pharmaceutically acceptable excipient.

A “subject” may be a vertebrate, mammal, or domestic animal. Hence, medicaments according to the invention may be used to treat any mammal, for example livestock (e.g., a horse), pets, or may be used in other veterinary applications. Most preferably, the subject is a human being.

A “therapeutically effective amount” of a tissue resident memory T cell, is any amount which, when administered to a subject, is the amount that is needed to produce the desired effect.

The amount of agent may be an amount from about 10\hat{ }5 T_{RM} cells to about 10\hat{ }7 T_{RM} cells, and most preferably from about 10\hat{ }5 T_{RM} cells to about 10\hat{ }6 T_{RM} cells.

A “pharmaceutically acceptable vehicle” as referred to herein, is any known compound or combination of known compounds that are known to those skilled in the art to be useful in formulating pharmaceutical compositions, specifically formulation for T-cell based therapies.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example, calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-28 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 28 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

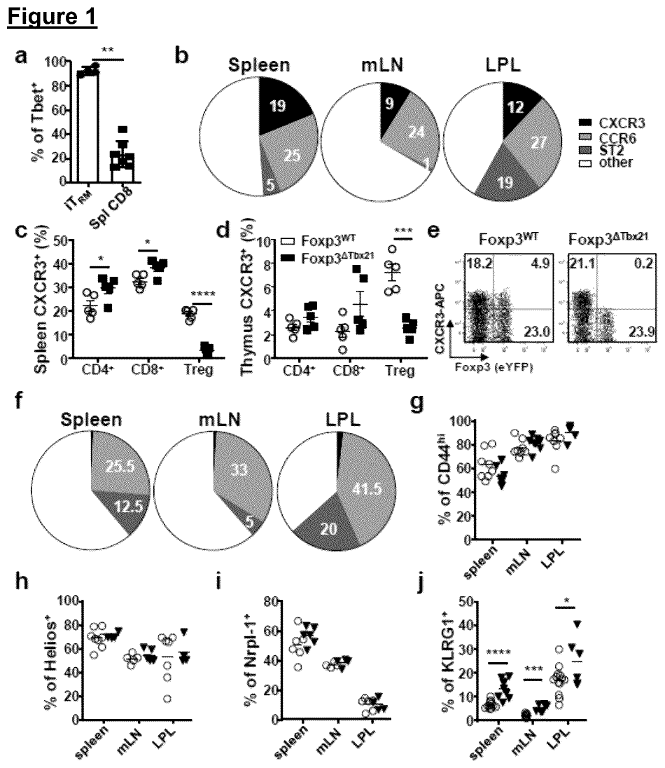

FIG. 1 shows Foxp3-dependent Tbx21 excision results in reduced number of type 1 Treg cells. (a) Percentage of intestinal T_{RM} or spleen CD8+ T cells stained for T-bet by flow cytometry analysis (n=4-7). Ex vivo flow cytometry analysis of T cell populations in Foxp3WT and Foxp3ΔTbx21 mice in indicated organs. (b,f) Proportion of Treg cells (CD4+Foxp3+) in the spleen, mesenteric lymph nodes (mLN) or lamina propria (LPL) expressing either CXCR3, CCR6 or ST2 in (a) Foxp3WT or (f) Foxp3ΔTbx21 mice (n=4-12). (c,d) Percentage of CXCR3 expressed in CD4+, CD8α+ and Treg cells in (c) spleen or (d) thymus (n=5). (e) Representative flow cytometry plot of CXCR3 expression in splenic CD4+ T cells. (g-j) Proportion of Treg cells expressing (g) CD44, (h) Helios, (i) Nrpl1 or (j) KLRG1 in Foxp3WT (open circles) and Foxp3ΔTbx21 mice (closed triangles) (n=4-9). Bars depict mean, error bars represent±SEM. For statistical analysis, Mann-Whitney U test, or multiple t test (g-j) was used. *P<0.05; **P<0.01; ***P<0.001; ****P<0.0001.

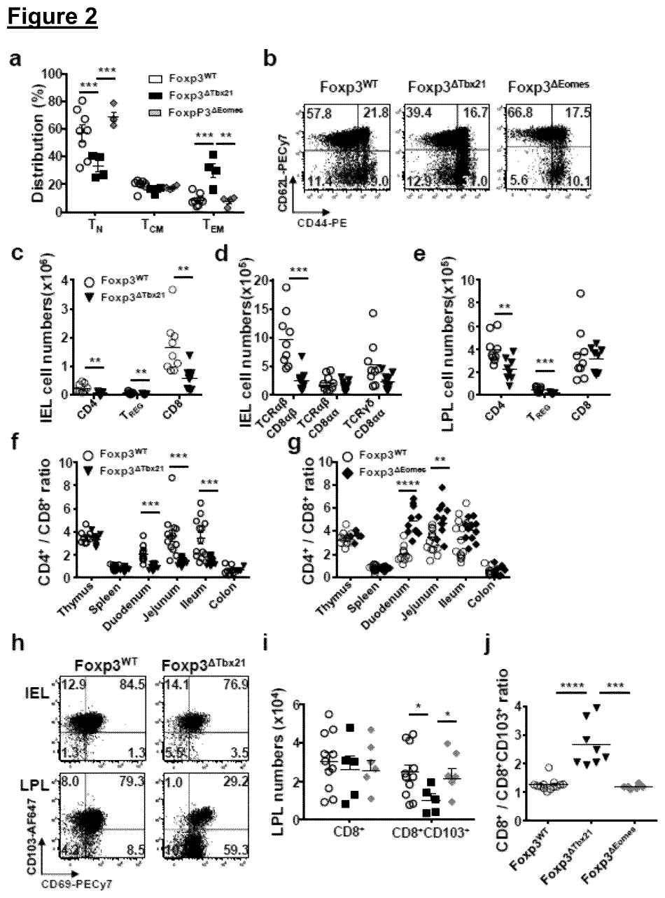

FIG. 2 shows Foxp3-dependent Tbx21 excision results in alterations in CD8 T cell 521 populations. (a, b) Flow cytometry analysis of CD8+ T cell populations in the spleen. (a) Percentage of naive (CD62LhiCD44lo), central memory (CD62LhiCD44hi) and effector memory (CD62L-CD44hi) CD8+ T cells in the spleen of Foxp3WT (open bars) n=8, Foxp3ΔTbx21 (closed bars) n=4, Foxp3ΔEomes mice (grey bars) (n=4). (b) Representative flow cytometry plot of CD62L and CD44 expression in spleen CD8+ T cells of indicated mouse lines. (c-e) Flow cytometry analysis of T cell populations, CD4+, Foxp3+ and CD8α+ T cells in intestinal compartments; (c) intraepithelial lymphocytes (IEL) in Foxp3WT (open symbols) or Foxp3ΔTbx21 (filled symbols) mice, (d) Numbers of indicated subpopulations of IEL of the same mice described under (c), and (e) the lamina propria (LPL) (n=8-9). (f, g) Ratios of CD4+Foxp3− and CD8+ T cells in indicated organs in Foxp3WT and (f) Foxp3ΔTbx21 or (g) Foxp3ΔEomes (n=4-12). (h) Representative dot plots showing CD103 and CD69 expression of IEL (top panels) or LPL (lower panels) CD8+ T cells of indicated mouse lines. (i) Cell numbers of total CD8+ and CD8+CD103+ T cells in the jejunum LPLs of Foxp3WT (open bars), Foxp3ΔTbx21 (closed bars) and Foxp3ΔEomes (grey bars) mouse lines (n=5-11). (j) Ratio of total CD8+ T cells over CD8+CD103+ T cells found in the LPL of indicated mouse lines. (n=6-13). Bars depict mean, error bars represent±SEM. For statistical analysis, multiple t test was used. *P<0.05; **P<0.01; ***P<0.001; ****P<0.0001.

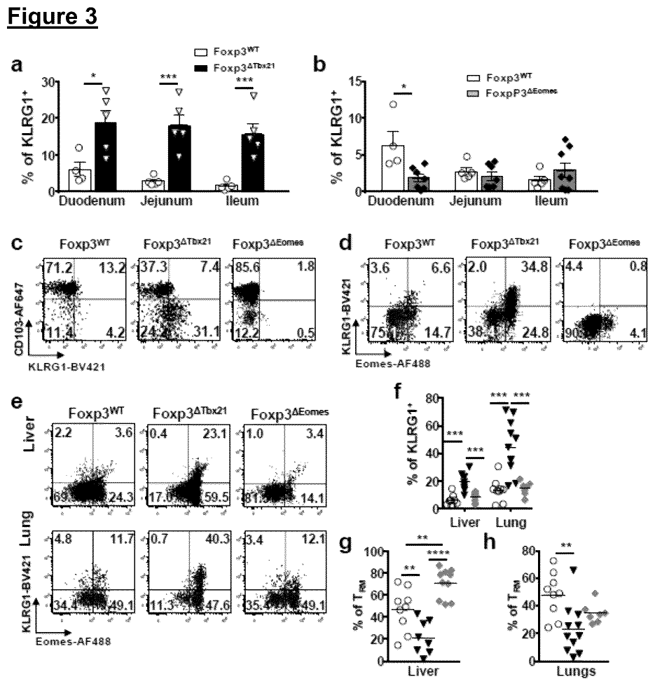

FIG. 3 shows reduced development of T_{RM} cells in absence of type 1 Treg cells. (a-d) Lamina propria lymphocytes were isolated from indicated small intestine sections of Foxp3WT (open bars), Foxp3ΔTbx21 (closed bars) and Foxp3ΔEomes (grey bars) mice and analysed by flow cytometry. (a,b) Cells were gated on TCRβ+CD8α+ and analysed for KLRG1 expression (n=4-8). (c,d) Representative flow cytometry plots showing (c) CD103 and KLRG1 expression or (d) Eomes and KLRG1 expression in TCRβ+CD4-CD8α+ LPLs of indicated mouse lines. (e-h) Representative plots (e) and cumulative data (f-h) showing proportion of TCRβ+CD4-CD8α+ cells which express KLRG1 in the liver and lungs of indicated mouse lines (f), proportion of T_{RM} cells in the (g) liver (CD69+Eomes-KLRG1−) and (h) lung (CD69+CD103+KLRG1−) (n=7-11). Bars depict mean, error bars represent±SEM. For statistical analysis, Mann-Whitney U test or multiple t test (f-h) was used. *P<0.05; **P<0.01; ***P<0.001; ****P<0.0001.

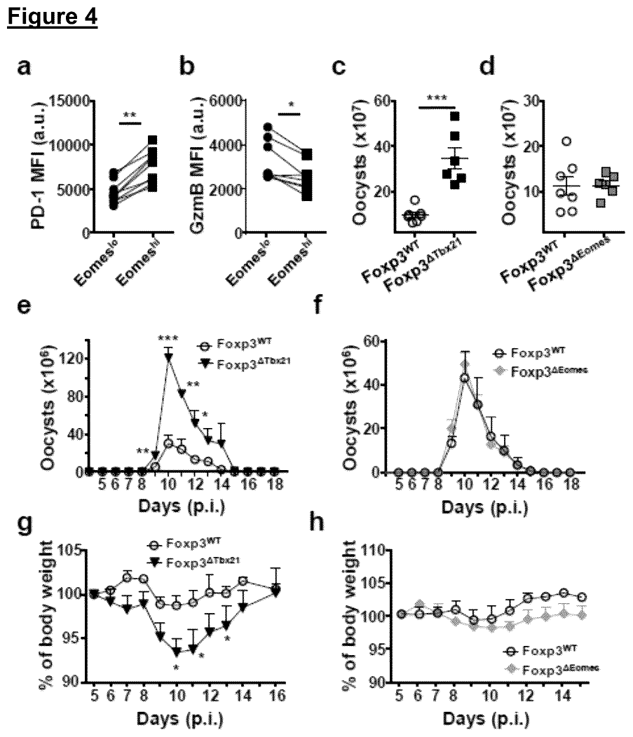

FIG. 4 shows reduced T_{RM} cell development results in increased susceptibility to infection. (a-b) TCRβ+CD8α+ lamina propria lymphocytes, Eomeshi or Eomeslo, from Foxp3ΔTbx21 mice were analysed by flow cytometry, 48 hrs after stimulation with 25 μg of anti-CD3 i.p., for (a) PD-1 and (b) GrzmB expression (n=8-9). (c-h) Foxp3WT (open symbols), (c,e,g) Foxp3ΔTbx21 (closed symbols) or (d,f,h) Foxp3ΔEomes (grey symbols) mice were orally infected with 1000 E. vermiformis oocysts. (c,d) Cumulative number of oocysts collected from individual mouse faeces from day 5-18. (e,f) Number of oocysts shed per day per individual mouse. (g,h) Body weight change during the course of E. vermiformis infection (n=3-4 per experiment, 2 biological repeats). Bars depict mean, error bars represent±SEM. For statistical analysis, multiple t test or Wilcoxon test (a,b) was used. *P<0.05; **P<0.01; ***P<0.001.

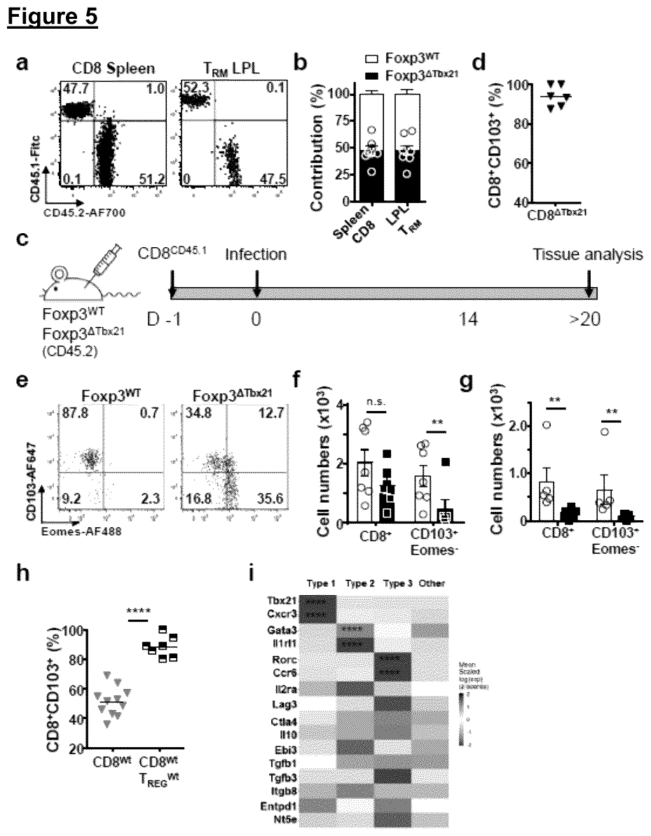

FIG. 5 shows recruitment of type 1 Treg cells determines T_{RM} cell differentiation. (a-b) Rag2-deficient mice were reconstituted with bone marrow from Ctrl (CD45.1) or Foxp3ΔTbx21 (CD45.2) mice. Contribution of each donor was assessed for total CD8 T cells in the spleen and the T_{RM} (CD8+CD103+KLRG1−) LPL population; (a) representative dot plots, (b) overview of individual mice assessed (n=8). (c) Description of adoptive transfer model; Foxp3WT or Foxp3ΔTbx21 mice received C57Bl/6 CD45.1+CD8α+ T cells intravenously, one day prior to oral infection with E.vermiformis. After infection resolution and on week 3-4 post-inoculation, lamina propria lymphocytes (LPL) were isolated from the small intestine and analysed by flow cytometry. (d) T_{RM} cell development as proportion of total CD8 T cell in the LPL of CD45.1 host mice transfer with splenic Foxp3ΔTbx21-derived CD8 T cells and subsequent E. vermiformis challenge as described under (c) (n=6). (e) Representative dot plot of lymphocytes pre-gated on CD45.1, followed by gating on TCRβ and CD8α and analysed for expression of CD103 and Eomes in indicated mouse lines. (f-g) Cell numbers of total CD8α+ LPLs and CD103+Eomes-CD8α+ LPLs obtained from small intestine on week (b) 3 or (c) 9 post infection. Gating performed as described in (a) (n=5-7). (h) Proportion of CD8α+CD45.1+ T cells recovered expressing CD103, after transfer of wild type CD45.1+CD8α+ T cells with or without wild type Treg cells into Foxp3ΔTbx21 mice and E. vermiformis challenge. (i) Single-cell RNA-sequencing analysis of Treg cell subtypes, organised by type 1 (85 cells), 2 (35 cells), 3 (28 cells) or other undefined Treg cells. Bars depict mean, error bars represent±SEM. For statistical analysis, Mann-Whitney U test was used. **P<0.01; ****P<0.0001, n.s.=none significant.

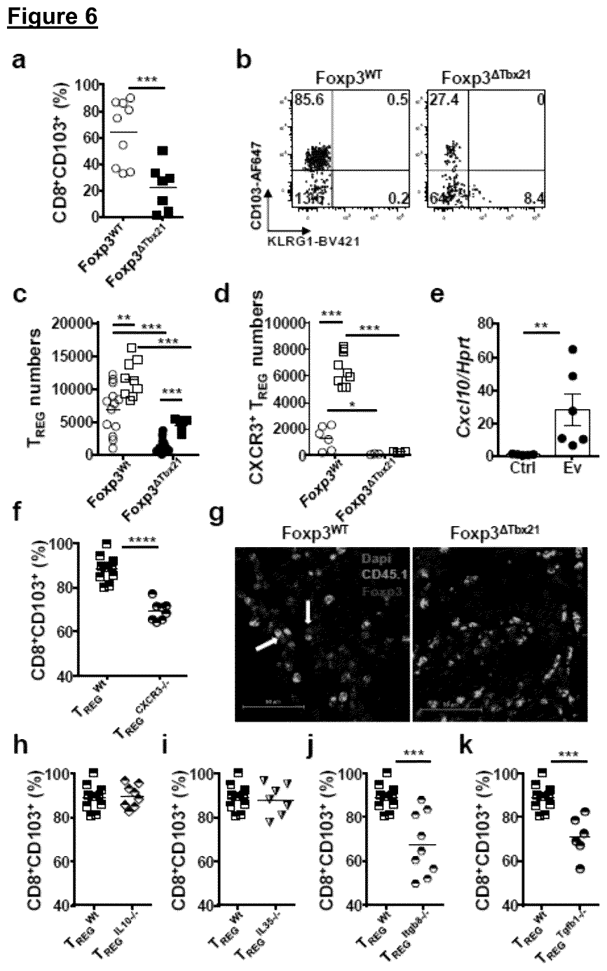

FIG. 6 shows type 1 Treg cells promote T_{RM} cell development via TGFβ availability. (a,b) Foxp3WT or Foxp3ΔTbx21 mice received C57Bl/6 CD45.1+CD8α+ T cells intravenously, one day prior to oral infection with Yersinia pseudotuberculosis. 2-3 weeks later lamina propria lymphocytes (LPL) were isolated from the small intestine and analysed by flow cytometry. (a) Proportion of CD8α+CD45.1+ T cells recovered expressing CD103 (n=7-9). (b) Representative dot plot of lymphocytes pre-gated on CD45.1, followed by gating on TCRβ and CD8α and analysed for expression of CD103 and Eomes in indicated mouse lines. (c-d) Ileum LPL Foxp3WT (open symbols) or Foxp3ΔTbx21 (closed symbols) mice were analysed at steady state (circle) or 10 days after E. vermiformis (Ev) (squares) infection, for (591 c) numbers of Treg, (d) number of Treg expressing CXCR3 (n=4-9). (e) C57BL/6 mice were infected or not with E.3 vermiformis and at day 10 Cxcl10 mRNA levels over Hprt were assessed in the ileum (2 594 biological repeats n=5-8). (f-k) Foxp3ΔTbx21 mice received C57Bl/6 CD45.1+CD8α+ T cells intravenously, one day prior to oral infection with E. vermiformis. On week 3 post-infection, LPL were isolated from the small intestine and analysed by flow cytometry for the expression of CD103. In addition to CD8CD45.1 cells, wild type Treg cells or Treg cells deficient in (f) CXCR3, (h) IL-10, (i) IL-35, (j) Integrinβ8 or (k) TGFβ1 were co-transferred. Mice receiving wild type T_{REG} cells were cumulated and used in panels (f, h-k). (g) Indicated mouse lines received CD45.1+CD8α+ T cells intravenously, one day prior to oral infection with E.vermiformis. On day 10 post-infection, small intestine were stained for CD45.1 (green), Dapi (blue) and Foxp3 (red). Representative immune histochemistry pictures (objective 40×, zoom 1.5×) shown from areas with oocysts (n=4). White arrows indicate close proximity of CD8 T cell and TREG. Bars depict mean, error bars represent±SEM. For statistical analysis, Mann-Whitney U test was used . . . *P<0.05; **P<0.01; ***P<0.001; ****P<0.0001.

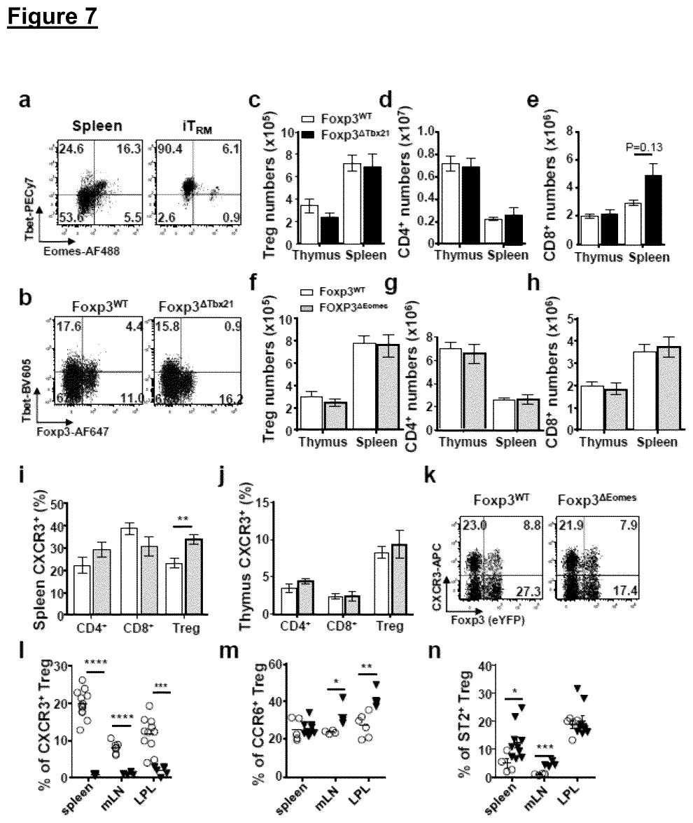

FIG. 7 shows Foxp 3 dependent Tbx21 conditional deletion results in reduced number of type 1 Treg cells Ex vivo flow cytometrically analysed cells showing a) Representative plots showing Tbet and Eomes expression by intracellular staining of CD8 cells in C57BL/6 spleen or intestinal T_{RM} population, b) representative plots of CD4 T cells stained for Foxp3 and Tbet. c-e) Number of Treg, CD4 and CD8 T cells in the thymus and spleen of Foxp 3 WT (open bars) and Foxp3ΔTbx 21 (closed bars) mice n=9. f-h) Number of Treg, CD4 T cells and CD8 T cells in Foxp3WT (open bars) and Foxp3ΔEomes (grey bars) mice i-j) Percentage of CXCR3 expression in CD 4 CD 8 α and Treg cells in i) spleen or j) thymus. Representative flow cytometry plot of CXCR3 expression in splenic CD4 T cells in Flow cytometric analysis of proportion of Treg cells in spleen, mesenteric lymph nodes mLN and lamina propria expressing CXCR3, CCR6 or ST2 (n≥4 open symbols Foxp3ΔWT closed Foxp3ΔTbx21 Error bars represent±SEM For statistical analysis, Mann Whitney U test or multiple t test h-m) was used P<0 05 P<0 01 P<0 001 P<0 0001.

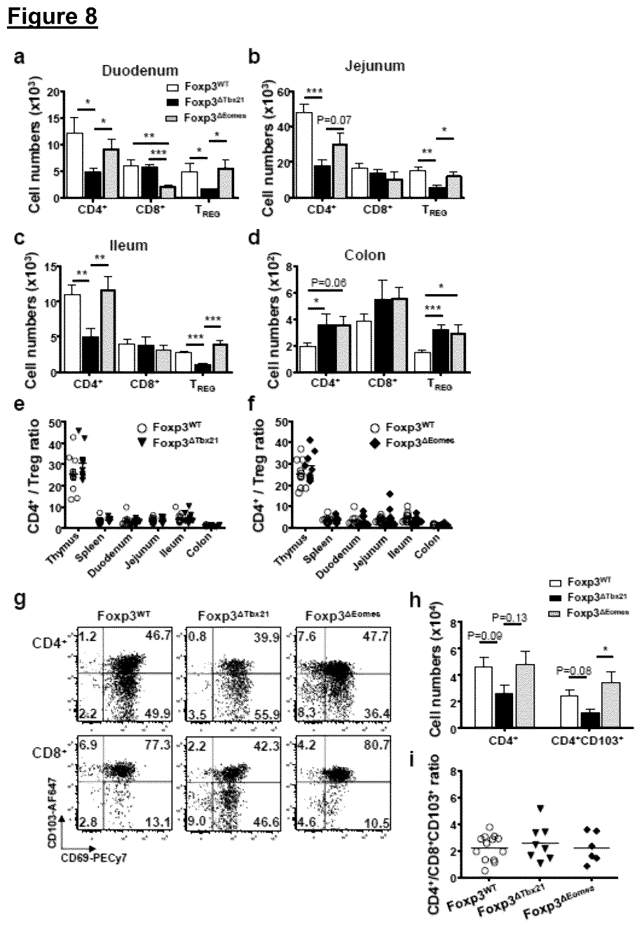

FIG. 8 shows Tbet or Eomes deficiency in Tregs is associated with alterations in T cell phenotype in the small intestine. Lamina propria lymphocytes (LPL) were isolated from Foxp3 WT, Foxp3ΔTbx21 and Foxp3ΔEomes mice and analysed by flow cytometry. a-d) Cell numbers in the LPL fraction of (a) duodenum (n=8-11), (b) jejunum (n=10-14), (c) ileum (n=9-13) and (d) colon (n=4-7) were determined by gating on TCRβ+ cells, followed by gating on CD4+CD8α−YFP−(CD4+), CD4+CD8α-YFP+ (Treg) and CD4-CD8α+ T cells (CD8+). e-f) Ratio between CD4+Foxp3− T cells and CD4+YFP+ T cells in individual animals from Foxp3WT (open circles) and e) Foxp3ΔTbx21 (closed triangles) or (f) Foxp3ΔEomes (closed diamonds) (n=8). g) Representative flow cytometry dot plots showing CD103 and CD69 staining of CD4+YFP− (top panels) or CD8+ (lower panels) LPL cells of indicated mouse lines. h) Number of total CD4+ T cells and CD103+CD4+ T cells in the lamina propria of Foxp3WT (open bars), Foxp3ΔTbx21 (closed bars) and Foxp3ΔEomes (grey bars) mouse lines (n=5-11). i) Ratio of Foxp3−CD4+ T cells/CD8α+CD103+ T cells found in the LPL of indicated mouse lines. (n=6-13). Error bars represent±SEM. For statistical analysis, Mann Whitney U test was used. *P<0.05; **P<0.01; ***P<0.001; *** P<0.0001.

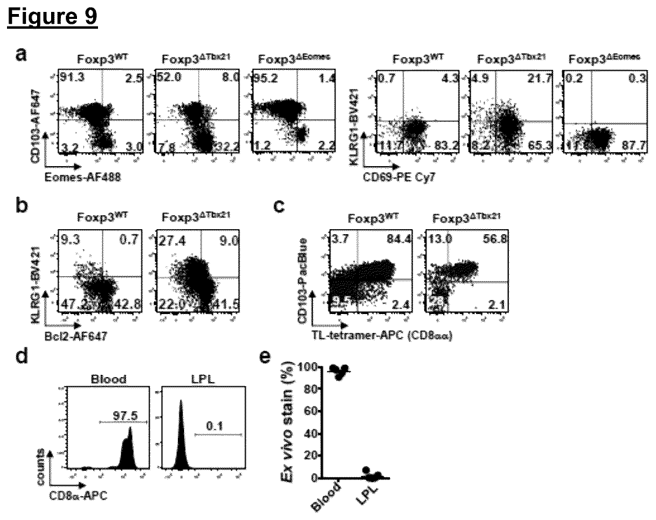

FIG. 9 shows absence of type 1 Tregs results in reduced T_{RM} cells Lamina propria lymphocytes were isolated from Foxp3WT, Foxp3ΔEomes and Foxp3ΔTbx 21 mice and examined by flow cytometry. a) Representative flow cytometry dot plots showing CD103 versus Eomes and KLRG1 versus CD69 in indicated mouse lines. b) Representative flow cytometry dot plots showing KLRG1 versus Bcl2 protein expression. c) Representative flow cytometry dot plots showing CD103 versus TL tetramer (CD 8αα staining) (n=4). d) Representative flow cytometry dot plots showing in vivo staining of CD8α in blood and LPL compartment and e overview graph (n=5) * P<0.05, **P<0.01, ***P<0.001, ****P<0.0001.

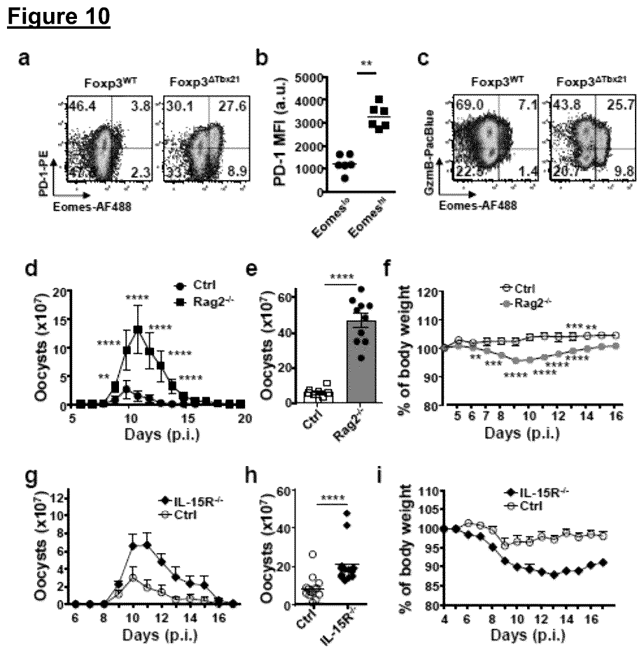

FIG. 10 shows reduced T_{RM} cell development increases infection susceptibility Lamina propria lymphocytes (LPL) or spleen CD8 T cells were isolated from Foxp3WT and Foxp3ΔTbx 21 mice 48 hrs after i.p. injection with anti CD38 antibodies and examined by flow cytometry. a-c) Representative flow cytometry dot plots showing (a) PD1 or (b) granzyme B versus Eomes protein expression in LPLs (n=4) b) As for a), but showing cumulative data for the spleen (n=6). d-i) C 57BL/6 (control) or (d-f) C57BL/6 Rag2^{-/-} of (g-i) IL 15 R mice were orally infected with 1000 E vermiformis oocysts (g) Daily feacal oocyst counts per individual mouse for indicated days, e,h) accumulative oocyst counts (days 6-17 f,i) changes in body weight during E vermiformis infection (n=5-9, 2 biological repeats) Error bars represent±SEM For statistical analysis, Mann Whitney U test (e,h) multiple t test (d,f,g,f) or Wilcoxon (b) was used *P<0.05, **P<0.01, ***P<0.001, ****P<0.0001.

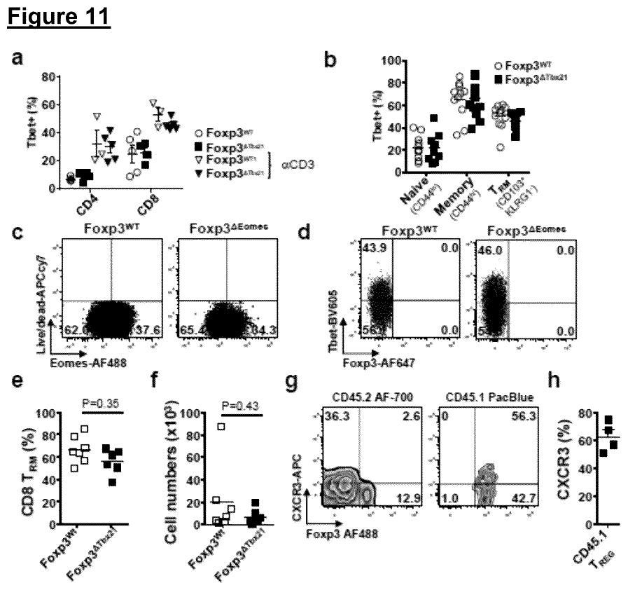

FIG. 11 shows Tbet expression and T_{RM} cell development upon immune activation (a-d) Indicated T cells were assessed by intracellular flow cytometry staining under steady state or 24 hrs after in vivo anti-CD3 stimulation. a) Overview of Tbet expression in Foxp3WT (open symbols) and Foxp3ΔTbx21 (closed symbols) splenic CD4 and CD 8 T cells. b) Tbet expression analysis by intracellular staining for splenic CD 8 naïve and memory T cells as well as LPL sourced T_{RM} cells (n=11 12). Representative plot of splenic CD 8 T cells from Foxp3WT and Foxp 3ΔEomes mice stained for (c) Eomes or (d) Tbet 24 hrs after activation (n=3 5). e-h) Foxp3WT (open symbols) and Foxp3ΔTbx21 (closed symbols) mice were adoptively transferred with CD8 CD45.1 T cells, upon which mice were challenged with E vermiformis a day later At the peak of infection, day 10 LPLs were isolated and CD45.1 were analysed by flow cytometry for (e) proportion of T_{RM} cells (CD8+CD103+Eomes−) and (f) total CD8+CD45.1+ T cells (n=67). (g) As under e), using CD45.2+Foxp3ΔTbx21 mice as hosts with additional CD45.1+Wt Treg cells transferred, showing representative flow cytometry plots of CD4+ T cells in the LPL, endogenous (CD45.2+) and transferred (CD45.1+) CD4 and Treg cells expressing CXCR3. h) Overview of CXCR3 proportions of transferred Treg cells as under f-g). For statistical analysis, Mann Whitney U test was used.

FIG. 12 shows T_{RM} cell development in selected mouse lines a,b) Lamina propria CD4 Foxp3 were isolated and enumerated of the ileum from Foxp3WT and Foxp3ΔTbx21 mice at steady state (circles) or 10 days after E vermiformis (squares) (n=5-8). c) Indicated mouse lines received CD45.1+CD8α+ T cells intravenously, one day prior to oral infection with Ex On day 10 post infection, small intestine were stained for CD45.1 (green) Dapi (blue) and Foxp3 (red). Representative immune histochemistry pictures (objective 20× zoom 15×, scale bar 50 μm shown, with indicated area enlarged O ocysts are characteristic round unstained areas, such as part indicated with *. An area without oocysts for comparison shown (3rd panel) f) Relative expression levels of splenic CXCR3. or CXCR3+ Treg cells (n=4). LPL were isolated from d,e) C57BL6 (control) or IL 10 deficient mice, g, j) or bone marrow chimeric mice generated with C 57BL6 mice or g, h) IL-35 deficient mice, g,i) Foxp3ΔItgβ8 mice, g,j Foxp3Δtgfβ1 mice. Representative flow cytometry plots are shown (f) and cumulative proportion of CD103 expressing cells (h-j) (n=7-10). Bars represent mean. For statistical analysis, Mann Whitney test was used ***P<0.001, ****P<0.0001.

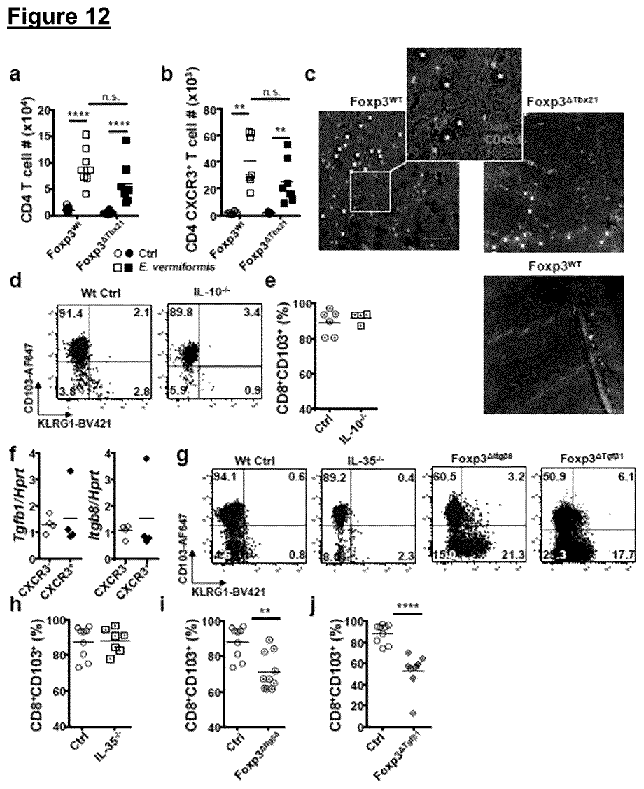

FIG. 13 shows flow cytometry analysis gating strategy. a) Representative flow cytometry plots from spleen of a C57BL/6 Foxp3ΔTbx21 mouse, showing lymphocyte gating, doublet exclusion and dead cell exclusion, followed by CD4 selection and Treg selection based on eYFP and tdRFP detection. b) Representative flow cytometry plots from LPL of a C57BL/6 Foxp3ΔTbx21 mouse, showing lymphocyte gating, doublet exclusion and dead cell exclusion, followed by inclusion of TCRβ+ CD69+ and CD8α+. In LPL, T_{RM} are defined as CD103+.

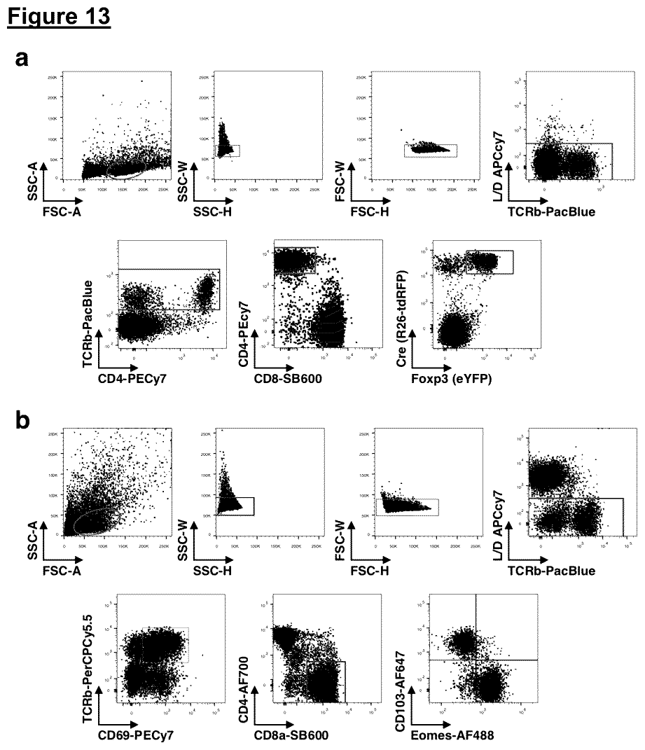

FIG. 14 shows RFP detection in Foxp3ΔTbx21 mice. a-c) Representative flow cytometry plots from spleen of a C57BL/6 Foxp3ΔTbx21 mouse, as in FIG. 13a, showing a) eYFP and tdRFP detection in the CD4 life gate, b) in the CD4 Foxp3 YFP population and c) the CD8 population Two representative plots are shown of 2 individual mice.

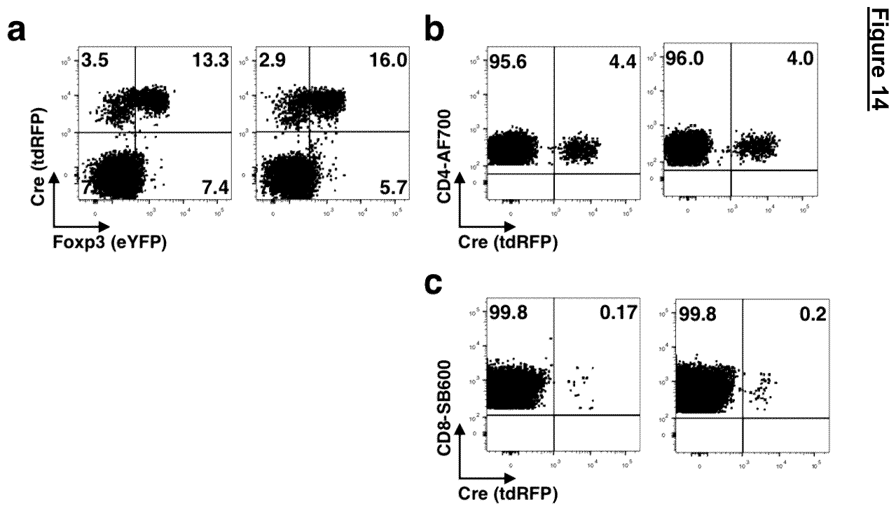

FIG. 15 shows the experimental layout detailing the production and testing of the T_{RM} cells, from the initial cell extraction from a mouse to the sorting of the cell population through a FACS machine.

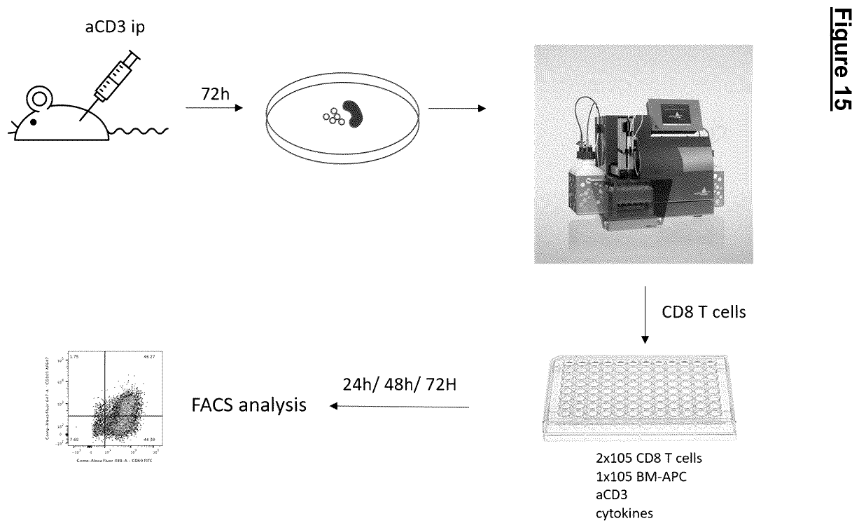

FIG. 16 shows that bone marrow derived dendritic cells (BMDC) maintain CD69 expression in CD8 T cells. When effector CD8 T cells are cultured with anti-CD3 in the presence of IL-15 and TGFβ alone (circles) or with BMDC (squares), the CD69 marker, critically expressed on T_{RM} cells remains expressed in the presence of BMDC, but is lost in the absence of BMDC.

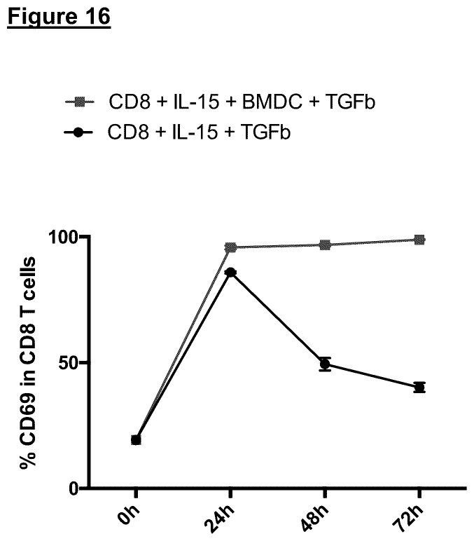

FIG. 17 shows that IL-7 induces CTLA-4 expression in T_{RM}. Two independent duplicate experiments show the difference in CTLA-4 expression when effector CD8 T cells are cultured in the absence of IL-7 (black) and with the addition of IL-7 (grey).

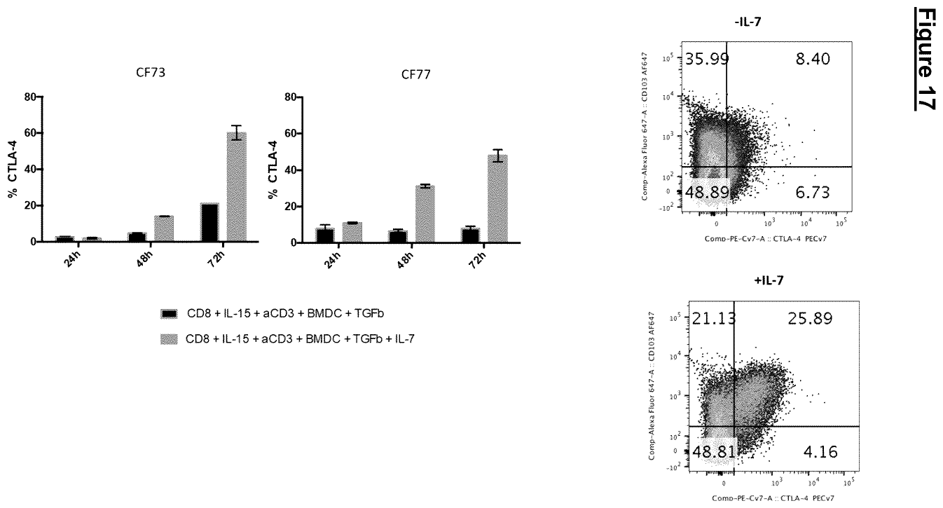

FIG. 18 shows that IL-2 also induces CTLA-4 expression in T_{RM}. The data show the expression of CTLA4 when effector CD8 T cells are cultured in the absence of IL-7 and IL-2 (black), with the addition of IL-7 (light grey), and with the addition of IL-2 (dark grey). Although a similar proportion of CD8 T cells expresses CTLA-4 if cultured with IL-7 or IL-2, the level of CTLA-4 expression is on average more robust for cultures containing IL-7 than for cultures containing IL-2.

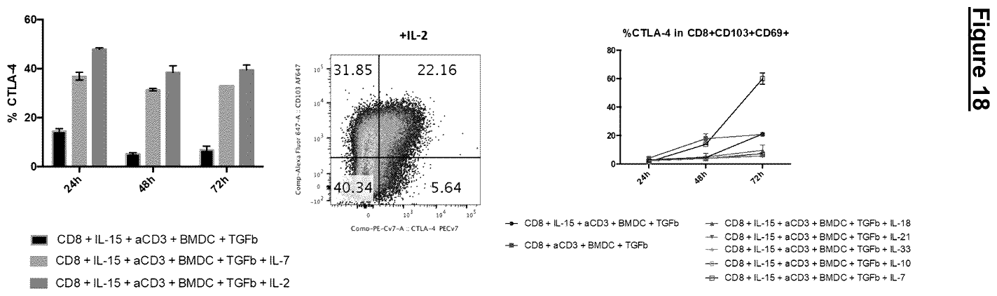

FIG. 19 shows the experimental layout detailing the production and testing of the T_{RM} cells, as well as the in vivo challenges and ex vivo analysis performed on the organs of the mice post-challenge. The inventor wanted to confirm whether the characteristics and phenotypes of the T_{RM} cells produced in vitro correlate with their in vivo seeding into the organs.

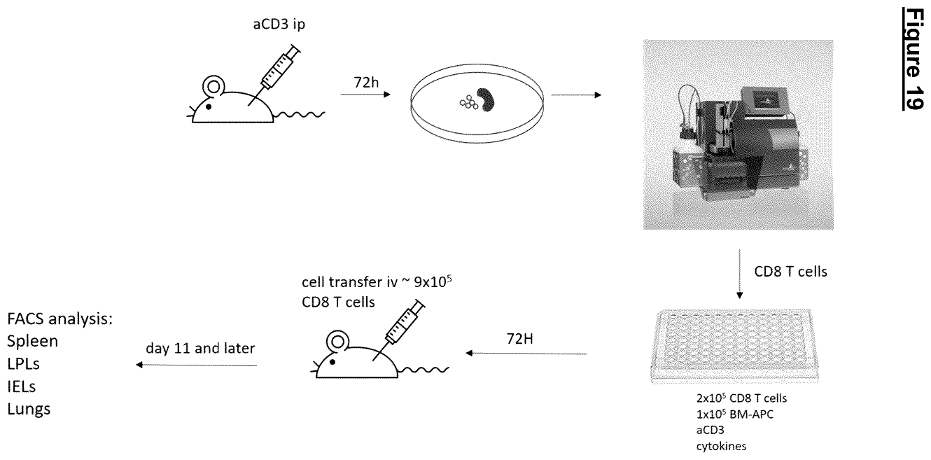

FIG. 20 shows the expression profile of the markers CD69, CD103 and CTLA-4 for the different T cells cultures used in in vivo challenges. The T cells used for the challenges were cultured under the following conditions or groups:
Group 1: CD8+BMDC+aCD3+ TGFb+IL-15=T_{RM};
Group 2: CD8+BMDC+aCD3+ TGFb+IL-15+IL-7=T_{RM}+IL-7;
Group 3: CD8+BMDC+aCD3+IL-15=effector; and
Group 4: CD8+BMDC+aCD3+ TGFb+IL-15+IL-2=T_{RM}+IL-2.

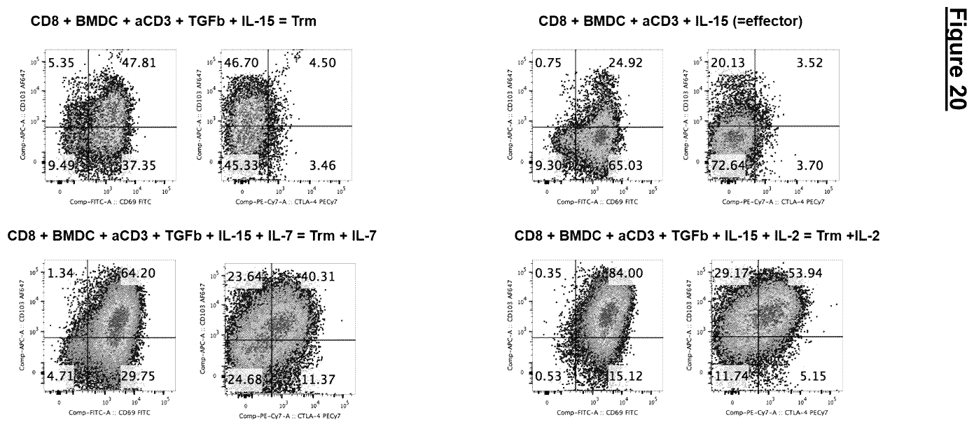

FIG. 21 shows the number of CD8+ T cells derived from the spleen of challenged mice over the course of 40 days. Effector CD8 T cells were cultured under the conditions indicated in FIG. 20 and transferred into a full C57BL6/J host. At indicated times, the presence of transferred cells (CD45.1) was assessed in the spleen. Graphs show two experiments pooled. Experiment 1 (black symbols) did not include Group 4 and analysis was performed at time point 11-14 only. Experiment 2 (grey symbols) included Group 4 and analysis was performed at several time points and only shown are 2 out of 6 mice in which cells were recovered at the late time point. The data shows that IL-7 cultured T_{RM} cells (group 2) were found in substantial numbers in comparison to T_{RM} cells cultured without IL-7.

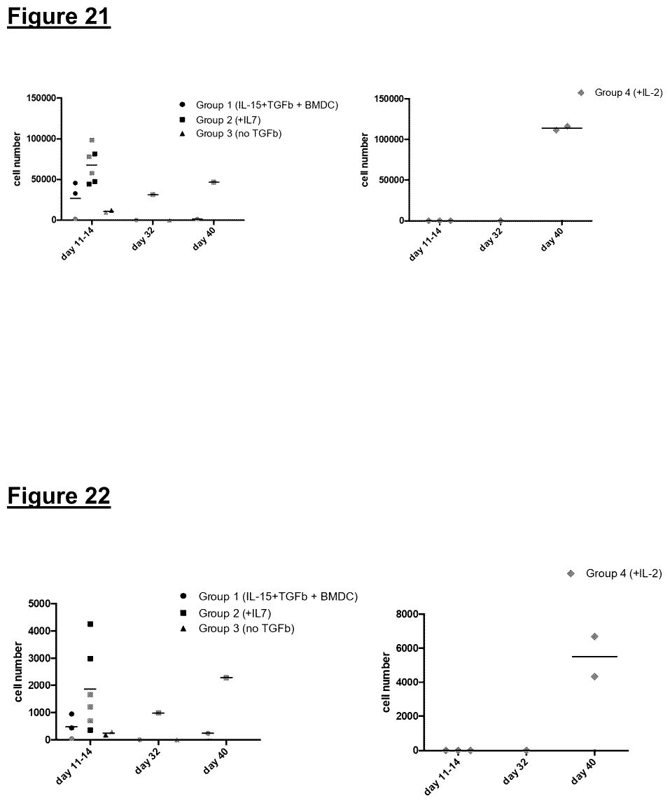

FIG. 22 shows the number of CD8+ T cells derived from the lamina propria of the intestine of challenged mice over the course of 40 days. Effector CD8 T cells were cultured under the conditions indicated in FIG. 20 and transferred into a full C57BL6/J host. At indicated times, the presence of transferred cells (CD45.1) was assessed in the lamina propria of the intestine. Graphs show two experiments pooled. Experiment 1 (black symbols) did not include Group 4 and analysis was performed at time point 11-14 only. Experiment 2 (grey symbols) included Group 4 and analysis was performed at several time points and only shown are 2 out of 6 mice in which cells were recovered at the late time point. The data shows that IL-7 cultured T_{RM} cells (group 2) were found in substantial numbers in comparison to T_{RM} cells cultured without IL-7.

FIG. 23 shows the number of CD8+ T cells derived from the lungs and IEL compartment of the intestine of challenged mice over the course of 32 days. Effector CD8 T cells were cultured under the conditions indicated in FIG. 20 and transferred into a full C57BL6/J host. At indicated times, the presence of transferred cells (CD45.1) was assessed in the lungs and IEL compartment of the intestine. Graphs show experiments (experiment 1-black symbols; experiment 2-grey symbols) in both organs showing the three groups and analysis was performed at several time points. The data shows that IL-7 cultured T_{RM} cells (group 2) were found in substantial numbers in comparison to T_{RM} cells cultured without IL-7.

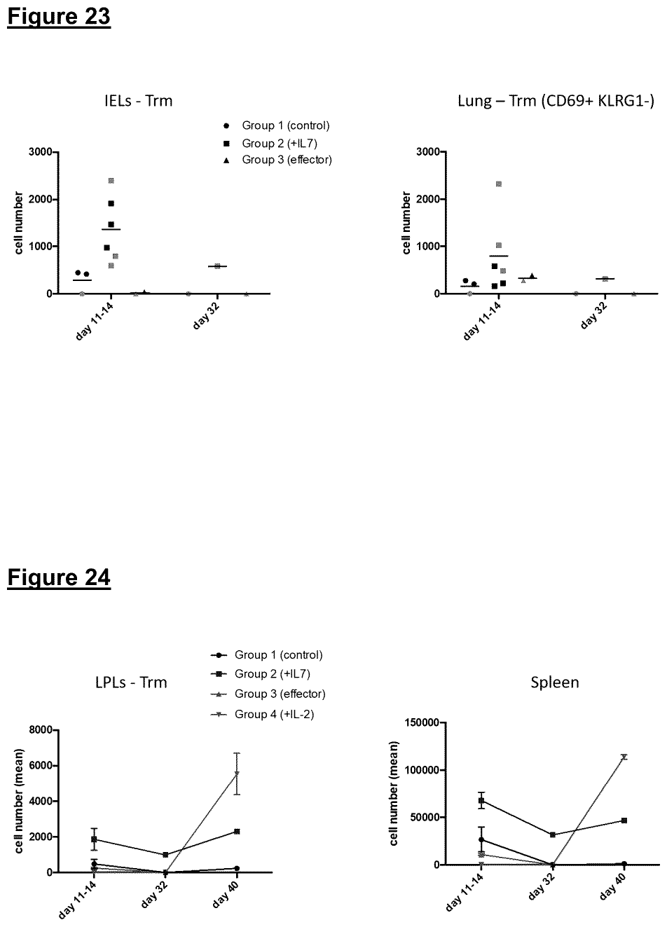

FIG. 24 shows the comparative number of CD8+ T cells derived from the lamina propria of the intestine and the spleen of challenged mice over the course of 40 days. Effector CD8 T cells were cultured under the conditions indicated in FIG. 20 and transferred into a full C57BL6/J host. At indicated times, the presence of transferred cells (CD45.1) was assessed in the spleen and the lamina propria of the intestine. The data shows that IL-7 cultured T_{RM} cells (group 2) were found in substantial numbers in comparison to T_{RM} cells cultured without IL-7.

### EXAMPLES

The inventors hypothesised that T_{REG} cells are important in the generation of T cells, T_{RM} cells, that are able to penetrate deeply into tissues and that are highly effective against solid tumours. The inventors aimed to determine the factors required to generate T_{RM} cells to enable the generation of T_{RM} cells in vitro with the ultimate aim to adapt current culture protocols to generate anti-tumour T cells to provide these with tissue penetrating properties to target both primary tumours and to provide critical organ-wide immunosurveillance directed against metastasis that have migrated to tissues away from the primary tumour. The inventors also aimed to assess whether the generation of T_{RM} cells was possible in the absence of T_{REG} cells in the medium as detailed in FIG. 15. Furthermore, the inventors assessed if the cells produced in vitro maintained their therapeutic properties, in particular, their ability to migrate and survive in vivo inside the tissues as detailed in FIG. 19.

### Materials and Methods

### Mice:

C57Bl/6J and C57Bl/6J CD45.1 mice were purchased from Charles River, France. Tbx21^{f/f} (Tbx21^{tm2Srnr}) and Eomes^{fl/fl} (Eomes^{tmtSrnr}) were kindly provided by Dr Reiner^{14, 82}, Foxp3eYFP-Cre (Foxp3^{tm4(YFP/icre)Ayr}) was kindly provided by Dr Rudensky⁸³, Rosa26-tdRFP was kindly provided by Dr Fehling⁸⁴, Rag2^{-/-}, IL15R^{-/-} (Jackson labs). Mice were bred at the Instituto de Medicina Molecular, Lisbon, Portugal. Male and female mice, aged and sex matched, at 8-18 weeks of age were used. Animals were housed in IVC cages with temperature-controlled conditions under a 12-hours light/dark cycle with free access to drinking water and food. All mice were kept in specific-pathogen-free conditions. All mice in the Foxp3eYFP-Cre Rosa26-tdRFP lines were stringently genotyped by PCR and those in which a knock out allele was detected were discarded (˜20%), appropriate Tbx21 presence was confirmed by blood typing for CD4 T cells expressing CXCR3. In addition, mice were counter screened for inappropriate expression of RFP in relation of eYFP (˜10% discarded) (FIG. 13-14). Bone marrow chimeras were generated by sublethal irradiation (450 rads) of Rag2-deficient mice and subsequent i.v. injection of bone marrow cells obtained. CXCR3^{-/-} (Cxcr3^{tm1Dgen})⁸⁵ were bred at the German Cancer Research Center (DKFZ), Heidelberg, Germany; IL-10^{-/-86} were bred at Instituto Gulbenkian de Ciência, Lisbon, Ebi3^{-/- 87} were bred at the institute for Immunology, University Medical Center Mainz, Germany, Itgb8f/f 88 and Tgfbif/f 89, crossed to Foxp3yfp-Cre were bred at the Immunology Virology and Inflammation department, Cancer Research Center of Lyon, France. All animal experimentation complied with regulations of the Direção-Geral de Alimentação e Veterinária Portugal and local ethical review committee and guidelines.

Cell isolation: Intestinal cells were isolated as previously described 90. Intestine was flushed with PBS to remove contents and opened longitudinally. After cutting into 1 cm pieces, it was incubated in PBS containing 20 mM Hepes, 100 U/ml penicillin, 100 μg/ml streptomycin, 1 mM Pyruvate, 10% FCS, 100 μg/ml polymyxin B and 10 mM EDTA for 30 min at 37° C. while shaking to release IELs. IEL single-cell suspensions were further purified using 37.5% isotonic Percoll. To isolate LPLs, intestinal tissue was then digested in IMDM medium containing 0.5 mg/ml of Collagenase D (Roche) and 0.2 mg/ml of DNaseI (Roche) for 25 min at 37° C. while shaking. Liver lymphocytes were isolated by mashing the organ through a 70 μm filter, followed by cell purification with 37.5% isotonic Percoll. Lungs were shredded in small pieces with scissors and digested in PBS containing 1 mg/ml Collagenase D, 37° C. during 30 minutes. The cell suspension containing the lymphocytes was obtained after passing through a 50 μm cell strainer.

Adoptive cell transfers: CD8α+ T cells and/or CD25+ cells (Treg) were purified from a single cell suspension of spleen and lymph nodes. Briefly, cells were labelled with anti-CD8α-APC or anti-CD25-APC antibody and selected with anti-APC MACS microbeads, according to the manufacturer's instructions. After counting, purity was determined by flow cytometry and cell numbers adjusted. To ensure a wide TCR diversity in the population transferred a minimum of 2×10⁶ CD8 T cells were used. Some of the recipient mice received in addition 0.4-1×10⁶ Treg cells. Infection was performed one day after cell transfer (day 0).

Infection challenges: Animals were infected with Eimeria vermiformis (Ev) as previously described in detail⁹¹. Briefly, oocysts were washed 3 times with deionized water, floated in sodium hypochloride and counted using a Fuchs-Rosenthal chamber. Mice received 500 oocysts of E.vermiformis by oral gavage in 100 μl of water and were analysed after the infection was cleared (from week 3 p.i). To determine burden of infection, animals were caged individually and faeces collected daily until oocysts were no longer detected. Animals were infected with Yersinia pseudotuberculosis (Yptb), kindly provided by Dr T. Bergsbaken, as previously described⁵⁸. Animals were infected with 10⁶ Yptb by oral gavage in 100 μl of water. Analysis of tissues was performed on days 15-19.

Flow cytometry: Single cell suspensions from spleen, lymph nodes, intestine, lung and liver were prepared and stained with antibodies (see list), according to the agreed standards⁹² and with indicated gating strategy (FIG. 13). In vivo staining were performed by i.v. injection of 3 μg of CD8α-APC antibody, whereupon mice were sacrificed 5 minutes later. TL-tetramer was kindly provided by NIH Tetramer Core Facility. Samples were run on a Fortessa X20 cytometer (BD Biosciences) and analysed with FlowJo software (TreeStar).

Quantitative RT-PCR: RNA was isolated using the Qiagen RNeasy Mini kit and cDNA generated using the High Capacity RNA-to-cDNA kit from Applied Biosystems. Amplification was performed using the SYBR Select Master Mix (Applied Biosystems) and the QuantiTect Primer Assays Mm_Cxcl10_1_SG, Mm_Tgfb1_1_SG, Mm_Itgb8_1_SG and Mm_Hprt_1_SG (Qiagen).

Immunohistochemistry and microscopy: Intestinal tissues were rolled into a “Swiss roll”, fixed in 10% formalin, rehydrated in 30% glucose and frozen in OCT media. Tissues were cut at 10 μm and sections treated with 4% paraformaldehyde. Blocking was performed using 10% BSA and the following antibodies were used for detection: CD45.1 (A20, Biolegend) and FOXP3 (FJK-16s, eBioscience). Slides were mounted in Fluoromount (Invitrogen) and imaged using a Zeiss LSM 880 microscope. Analysis was performed using Fiji software.

scRNA-Seq analysis Original data was produced and analysed in55. From the initial data-set, T_{REG} cells were selected based on Foxp3, excluding Tmems, stressed and low-quality cells. In order to analyse this subset, we followed a similar approach as⁵⁵, using the R package Seurat⁹³. Normalization of the data using the “LogNormalize” method and using a scale factor of 10⁵; and scale the data based on Negative Binomial Model and using UMI's. Subtypes of T_{REG} were defined using the following criteria: Type 1 (Cells with raw counts assigned to the genes Tbx21, Stat1 and Cxcr3); Type 2 (Cells with raw counts assigned to the genes Gata3, Stat6 and Il¹rl1); Type 3 (cells with raw counts assigned to the genes Rorc, Stat3 and Ccr6); other (Cells with no raw counts assigned to the genes Tbx21, Gata3 and Rorc).

### In Vitro Cultures:

Effector CD8 T cells were obtained from C57BL6/J or CD45.1 C57BL6/J mice previously i.p. injected with 25 μg anti-CD38. Cells were isolated via AutoMACS bead selection and cultured at 200.000 cells per flat bottom 96-well plates in IMDM medium. 100.000 BMDC, cultured via standard protocol using GM-CSF were added in indicated conditions. Cells were restimulated with 0.25 μg/ml anti-CD38, 10 ng/ml IL-15, and 0.5 ng/ml TGFβ, and where indicated 10-20 ng/ml IL-2 or IL-7. Cells were grown for 3 days before analysis or adoptive transfers into full C57BL6/J mice to test for tissue homing. Cells were assessed for the T_{RM} markers CD69, CD103, the absence of KLRG-1, and expression of CTLA-4.

In vitro differentiated cells were transferred into mice through intravenous injection. At the indicated time points, animals were sacrificed and lymphocytes from spleen, lungs and small intestine (both IEL and LPL fractions) were isolated following standard methods. Cell populations were analysed by flow cytometry. Transferred cells were distinguished from endogenous cells by their expression of the congenic marker CD45.1 and cell counts were performed using flow cytometry counting beads.

### Example 1

### Results

Deletion of Tbx21 in FoxP3⁺ Cells Reduces Type 1 T_{REG} Cells

T_{RM} cells express T-bet but not Eomes (FIG. 1a, FIG. 7a) 37. T_{REG} cells express lineage-associated chemokine receptors in different tissues, with immune type 1, 2 and 3 characteristics (FIG. 1b). To test whether T_{REG} cells expressing T-bet or Eomes influence T_{RM} cells, the inventors made use of the Foxp3^{eYFP-Cre} Tbx21^{fl/fl} Rosa26^{tdRFP/tdRFP} and Foxp3^{eYFP-Cre} Eomes^{fl/fl} Rosa26tdRFP/tdRFP mouse lines (referred to as Foxp3^{ΔTbx21} and Foxp3^{ΔEomes} respectively) and control Foxp3^{eYFP-Cre} Rosa26^{tdRFP/tdRFP} line (Foxp3^{WT}) (methods, FIG. 14). T-bet and Eomes activate the transcription of genes important in type 1 immune responses, such as the chemokine receptor CXCR3, trans-activated by T-bet³⁸. In line with enhanced type 1 inflammation in the absence of T-bet in T_{REG} cells^{35, 39} (FIG. 7b), spleen, but not thymus, showed proportional increases in CXCR3⁺CD4⁺ and CXCR3⁺CD8⁺ T cells in Foxp3^{ΔTbx21} mice (FIG. 1c-d). Numbers of CD4⁺Foxp3− or T_{REG} (CD4⁺Foxp3⁺) cells in thymus or spleen were similar (FIG. 7c-h), but spleen CD8⁺ T cells showed an increased trend in Foxp3^{ΔTbx21} animals (FIG. 7e)^{29, 35, 39}. No signs of autoimmunity in mice up to three months of age were observed.

The inventors confirmed that Foxp3-specific targeting, CD4⁺CXCR3⁺Foxp3−, but not CD4⁺CXCR3⁺Foxp3⁺, T cells were present in Foxp3^{ΔTbx21} mice (FIG. 1c-e), but observed an increase in the proportion and numbers of CXCR3⁺ T_{REG} cells in peripheral lymphoid organs of Foxp3^{ΔEomes} mice (FIG. 7i-k). The excision of T-bet in T_{REG} cells resulted in altered distribution, but not numbers, of T_{REG} subsets, with an increase in type 3 T_{REG} cells (FIG. 1f, FIG. 7l-n). T_{REG} cell populations in the LPL showed a more activated phenotype compared with those present in the secondary lymphoid organs (SLO), expressing higher levels of CD44 (FIG. 1g). Neuropilin-1 (Nrpl-1) and transcription factor Helios, were present mainly in SLO T_{REG} cells but reduced in the intestine. T_{REG} cells show a similar phenotype in Foxp3^{ΔTbx21} compared with Foxp3WT control mice (FIG. 1h-i) 29, the co-inhibitory receptor killer-cell lectin like receptor G1 (KLRG1), expressed on effector T cells and T_{EM} cells^{40, 41}, is increased on T_{REG} cells from Foxp3^{ΔTbx21} compared with Foxp3WT control mice (FIG. 1j). Collectively, these data show that in the absence of T-bet-expressing T_{REG} cells, the number of T_{REG} cells and their phenotype remains similar, but with alterations in proportions of T_{REG} subsets.

### Excision of Tbx21 or Eomes in T_{REG} Cells Alters CD8 T Cell Distribution

The splenic CD8⁺ T cell compartment of Foxp3^{ΔTbx21} mice show an increase in effector (T_{eff})/T_{EM} T cells (FIG. 2a-b). However, the intestinal intraepithelial fraction in Foxp3^{ΔTbx21} mice show a reduction in CD4 and CD8 T cells compared with Foxp3^{WT} controls (FIG. 2c). Within the CD8 IEL population the marked decrease in Foxp3^{ΔTbx21} mice is observed within the induced CD8αβ⁺, but not in the natural CD8αα⁺ IEL populations (FIG. 2d). The lamina propria (LP) compartment in Foxp3^{ΔTbx21} mice shows a reduction in CD4⁺ T cells, but not CD8⁺ T cells compared with Foxp3WT controls (FIG. 2e). This difference is apparent throughout all intestinal sections but the colon (FIG. 8a-d), in which CXCR3 and T-bet expression in T_{REG} cells is disjointed⁴².

Despite altered numbers of CD4⁺Foxp3-T cells and T_{REG} cells in the LPL compartment, and irrespective of the Foxp3-dependent excision of Tbx21 or Eomes, the proportion of CD4⁺ T cells and T_{REG} cells remains stable (FIG. 8e-f). However, the T cell population of Foxp3^{ΔTbx21} animals shows a marked proportional skewing towards CD8 T cells (FIG. 2f), while the opposite is observed in Foxp3^{ΔEomes} animals, particularly in the proximal intestine (FIG. 2g). These data indicate that the absence of T-bet or Eomes in T_{REG} cells, does not alter the proportional distribution between CD4⁺Foxp3⁺ and CD4⁺Foxp3⁻ T cells, but has a marked impact on the proportion of CD8⁺ T cell subsets in the small intestine.

Tbx21⁺ and Eomes⁺ T_{REG} Cells Influence the CD8 T Cell Memory Compartment

The reduction of T_{RM} cells in the intestine and increased proportion of circulating effector/T_{EM} cells in the absence of T-bet-sufficient T_{REG} cells suggested a potential role for these cells in the generation or maintenance of T_{RM} cells. Although there is a reduction in IEL numbers (FIG. 2c-d), all CD8⁺ IELs express the T_{RM} cell markers CD103 and CD69 (FIG. 2h). The LPL compartment in Foxp3^{ΔTbx21}, Foxp3^{ΔEomes} and Foxp3^{WT} mice were similar with respect to CD4⁺Foxp3⁻ T cells, which express high levels of CD69 with about half co-expressing CD103 (FIG. 8g). Although the inventors did not observe a difference in the phenotype of CD4 T_{RM} cells, Foxp3^{ΔTbx21} animals showed an overall trend in reduced numbers of CD4⁺Foxp3⁻ T cells and CD4⁺CD103⁺ cell numbers (FIG. 8h).

In the LPL compartment of Foxp3WT and Foxp3^{ΔEomes} animals, most CD8 T cells express the TRIM markers CD69 and CD103 (FIG. 2h-i). In contrast, in Foxp3^{ΔTbx21} animals, over half of the CD8+ T cells do not express CD103 (FIG. 2h-i). Therefore, despite similar total numbers of CD8+ T cells, in the absence of T-bet-expressing T_{REG} cells, the numbers of CD8+ T_{RM} cells are reduced in the intestine of these animals, with high proportional contribution of effector over TRY cells (FIG. 2i-j) resulting in a constant ratio between CD4 T cells and CD8 T_{RM} cells in all three mouse lines (FIG. 8i). These data indicate that immune networks in the intestine are fine-tuned, with increased CD8+ T cell ratios observed in the Foxp3^{ΔTbx21} animals possibly due to the accumulation of CD8+ effector T cells failing to develop into T_{RM} cells.

Tbx21⁺ T_{REG} Cells Influence T_{RM} Cell Development in Multiple Tissues

Upon skin infections, KLRG1⁺CD103⁻CD8⁺ effector T cells have been reported in the dermis early, but not late, nor in the epidermis¹⁹. In agreement with the population of CD103⁻CD8⁺ T cells observed in the small intestine of Foxp3^{ΔTbx21} animals under steady state conditions, a marked population of KLRG1⁺CD8⁺ T cells, around 20% of the total CD8 T cell population, in all sections of the small intestine was observed (FIG. 3a). In contrast, Foxp3ΔEomes animals, which harbour increased CXCR3⁺ T_{REG} cells (FIG. 7h), showed a reduction in KLRG1⁺CD8⁺ T cell numbers in the proximal intestine (FIG. 3b).

Co-staining with CD103 confirmed the KLRG1 protein to be expressed in a mutually exclusive form with CD103^{19, 26, 43}, and primarily present in Foxp3^{ΔTbx21} mice (FIG. 3c). The CD103-KLRG1⁺CD8 T cells present in Foxp3^{ΔTbx21} animals expressed high levels of Eomes, whereas few were found in Foxp3^{WT} and even less in Foxp3^{ΔEomes} animals (FIG. 3d, FIG. 9a). In agreement with their effector status, KLRG1⁺Eomes⁺CD8 T cells in Foxp3^{ΔTbx21} animals had reduced expression levels of the pro-survival protein Bcl-2, upregulated during T_{RM} cell maturation^{44, 45} (FIG. 9b). Furthermore, the proportion of cells expressing CD8αα homodimers, expressed in conjunction with CD8αβ heterodimers and characteristic for epithelial memory CD8 T cells⁴⁶, was reduced in Foxp3^{ΔTbx21} animals compared with Foxp3^{WT} controls (FIG. 9c). Lastly, in vivo staining confirmed that the majority of cells isolated from the LPL compartment did not recently circulate (FIG. 9d,e). Taken together, and without wishing to be bound to any particular theory, these data show that in the absence of T-bet expressing T_{REG} cells, CD8⁺ effector T cells accumulate at the intestinal barrier where they do not progress towards T_{RM} cells.

The presence of T_{RM} cells is described in many tissues⁴. Consistent with results in the intestine, the liver and lungs of Foxp3^{ΔTbx21} mice contained an increased proportion of effector CD8 T cells, expressing high levels of KLRG1 and Eomes, compared with Foxp3^{ΔEomes} and Foxp3^{WT} animals (FIG. 3e-f). Because CD103 expression is not considered a sufficient marker of T_{RM} cells in the liver, the inventors assessed the proportions of CD8+CD69+ cells negative for KLRG1 and Eomes. Foxp3^{ΔTbx21} mice contained fewer T_{RM} cells compared with Foxp3^{ΔEomes} and Foxp3^{ΔWT} mice in the non-lymphoid tissues assessed (FIG. 3g-h). Without wishing to be bound to any particular theory, these data suggest that type 1 T_{REG} cells are important in the generation of T_{RM} cells in multiple tissues.

### Compromised T_{RM} Cell Compartment Reduces Protection Against Pathogen Invasion

The inventors hypothesised that reduced T_{RM} cell numbers in Foxp3^{ΔTbx21} mice could reduce protection against new infections, since bystander-mediated activation of T_{RM} cells is an important defence mechanism limiting pathogen invasion^{47, 48,49}. We tested the acute response of intestinal CD8+ T cells by administrating anti-CD3 antibody upon which the LPL T cell response was assessed two days later. Eomes⁺CD8 T cells displayed a reduced activity profile compared with Eomes CD8 T cells with increased expression of PD-1 and reduced granzyme B (FIG. 4a,b, FIG. 10a-c)⁵⁰.

Next, the inventors challenged mice with the intracellular protozoan parasite Eimeria vermiformis (Ev), which infects murine small intestinal epithelial cells. In this infection model lymphocytes reduce parasite burden (FIG. 10d-f), with CD8+ T cells and IFNγ playing an important role in the clearance^{10, 51, 52}. Although type 1 T_{REG} cells were absent, which could be expected to lead to enhanced T cell-mediated immunity⁵³, in fact Foxp3^{ΔTbx21} mice showed impaired control of Ev infection compared to Foxp3^{WT} and Foxp3^{ΔEomes} animals (FIG. 4c-f). In contrast to mice devoid of lymphocytes (FIG. 10d-e), but in line with IL-15Rα-deficient mice required for T_{RM} cell survival²⁶, Foxp3^{ΔTbx21} mice, in which more effector cells are present but with an exhausted phenotype, lost body weight compared with Foxp3^{WT}, Foxp3^{ΔEomes}, and Rag2^{-/-} animals (FIG. 4g-h, FIG. 10f). These data are in line with T_{RM} cells offering protection against the apicomplexan parasite Plasmodium in the liver⁴⁹ and virus in the skin²⁶.

Type 1 T_{REG} Cells Enhance T_{RM} Development

T cells from Foxp3^{ΔTbx21} or Foxp3^{ΔEomes} mice where indistinguishable from Foxp3WT controls with respect to the expression of Tbet or Eomes, at steady state, upon activation or upon T_{RM} cell establishment (FIG. 11a-d). To assess if the accumulation of effector T cells and reduction of T_{RM} cells in tissues of Foxp3^{ΔTbx21} animals (compared to Foxp3WT) was CD8 T cell intrinsic, we generated mixed bone marrow chimeras with CD45.1 controls and Foxp3^{ΔTbx21} mice. The T_{RM} cells found showed similar contribution from both donors (FIG. 5a-b). Furthermore, we transferred CD8 T cells sourced from Foxp3^{ΔTbx21} mice (CD45.2) into control CD45.1 animals. A day later, mice were challenged with Ev, which is cleared after two weeks⁵⁴. The development of CD8^{CD45.1}CD103⁺ T_{RM} cells was assessed a week after parasite clearance, when T_{eff} cells have diminished (FIG. 5c). Within the transferred CD45.2⁺ population T_{RM} cells developed with high efficiency (FIG. 5d). Collectively, these results suggest a CD8 T cell extrinsic defect in Foxp3^{ΔTbx21} mice that inhibits the generation of T_{RM} cells. To confirm this, we transferred CD45.1⁺CD8⁺ T cells (CD8^{CD45.1}) into CD45.2⁺ Foxp3^{ΔTbx21} or Foxp3^{WT} animals. At the peak of infection (day 10), T_{RM} cells and effector T cells are present in the LPLs (FIG. 1l,f). In Foxp3^{WT} hosts, the majority of transferred CD8^{CD45.1} cells showed a characteristic T_{RM} cell profile of CD103 expression with low Eomes levels (FIG. 5e). In contrast, CD8^{CD45.1} T cells transferred into Foxp3^{ΔTbx21} hosts showed partial T_{RM} cell formation with a majority of these cells showing an effector phenotype, with expression of Eomes and absence of CD103 (FIG. 5e). Accumulated numbers of transferred CD8^{CD45.1} T cells were similar in the Foxp3^{ΔTbx21} hosts compared with the Foxp3^{WT} hosts, with reduced T_{RM} cells in the former (FIG. 5f). Analysis of mice nine weeks post Ev infection showed reduced numbers of detectable transferred CD8^{CD45.1}T cells overall in the Foxp3^{ΔTbx21} animals (FIG. 5g). These data confirm that the impaired CD8⁺ T_{RM} cell differentiation observed in the Foxp3^{ΔTbx21} animals is extrinsic to the CD8⁺ T cell population. The inventor's transfer system enabled the testing of the hypothesis that T_{REG} cells facilitate the development of T_{RM} cells via concomitant transfer of CD8^{CD45.1} T cells and T_{REG}^{WT} cells into Foxp3^{ΔTbx21} animals (FIG. 11g). In support of the hypothesis, the generation of T_{RM} cells in Foxp3^{ΔTbx21} animals was restored to levels observed in Foxp3^{WT} controls in the presence of control T_{REG} cells (FIG. 5h).

To understand if T-bet-expressing T_{REG} cells have specific functional attributes that may explain their role in TRY development, the inventors made use of a recent publically available set of single T_{REG} cell sequencing data⁵⁵. In line with previous reports^{42, 56}, although small trends may exists, the inventors did not uncover significant differences in T_{REG} cell effector molecules such as IL-10, IL-35, TGFβ, CD25, LAG3 or CTLA-4 across T_{REG} cell subsets defined by the presence of the characteristic lineage transcription factors Tbx21, Gata3 or Rorc (FIG. 5i). Furthermore, T-bet-deficient T_{REG} cells have been reported to show similar suppressive capacity as control T_{REG} cells^{35, 57}.

T_{RM} Development Relies on T_{REG} Recruitment to Make TGFβ Bio-Available Locally

The inventor's observations relied on the microbial presence under specific pathogen free conditions and the intracellular small intestinal parasite Ev, which provokes a very local response. The inventors made use of our CD8^{CD45.1} T cell transfer system (FIG. 5c), challenging the mice with the bacterium Yersinia pseudotuberculosis (Yptb), reported to induce T_{RM} cells^{58,59}. In line with results obtained using Ev, Yptb challenge resulted in efficient T_{RM} cell development in Foxp3^{WT} animals that was markedly reduced in Foxp3^{ΔTbx21} animals (FIG. 6a,b).

The transfer model of CD8^{CD45.1} T cells into Foxp3^{ΔTbx21} mice enabled the inventors to investigate the contribution T_{REG} cells make to promote T_{RM} cell development. In the absence of T-bet, T_{REG} cells express similar levels of CD103, CCR6 and P-selectin³⁵, but are unable to express CXCR3, important for localisation of T cells to areas of infection in non-lymphoid tissues^{7,35,39,60}. The local inflammatory environment controls recruitment of T_{RM} precursor cells^{58, 61}, and T_{REG} cells (FIG. 6c). Importantly, the T_{REG} cells recruited upon Ev infection predominantly show a type 1 phenotype, expressing CXCR3 (FIG. 6d, FIG. 11g-h). Taking into account the absence of alterations in effector and T_{RM} cells in the colon of Foxp3^{ΔTbx21} mice, where expression of CXCR3 is not T-bet dependent, the inventors hypothesised that T-bet expression in a subpopulation of T_{REG} cells facilitates the recruitment of these cells to the site of infection and brings them in close proximity with TRY precursor cells. Conform this, Foxp3-dependent excision of Tbx21 resulted in reduced recruitment of T_{REG} cells upon Ev infection (FIG. 6c), largely due to those expressing CXCR3 (FIG. 6d). In agreement with recruiting type 1 T_{REG} cells, the inventors found increased expression of the CXCR3 ligand, CXCL10 in intestinal tissues upon Ev infection (FIG. 6e). CD4 T cell numbers are reduced under steady state conditions in the LPL compartment of Foxp3^{ΔTbx21} mice (FIG. 1e), and could play an additional role in T_{RM} cell generation. However, Ev infection resulted in robust recruitment of CD4 T cells to the LPL compartment with a predominant T helper 1 phenotype (FIG. 12a,b). Making use of the transfer system (FIG. 5a), upon concomitant transfer with CD8^{CD45.1} cells, CXCR3-deficient T_{REG} cells were unable to support efficient development of T_{RM} cells compared with CXCR3-sufficient controls (FIG. 6f).

Aggregates of CD8⁺ and CD4⁺ T cells together with other immune cells such as macrophages and dendritic cells but without B cells, in areas of microbial invasion are commonly observed^{58, 62, 63, 64, 65}. Interactions between CD4⁺ and CD8⁺ T cells, although not required for T cell maintenance⁵⁹, likely constitute distinct microenvironments that may support T_{RM} differentiation. In line, the inventors frequently observed transferred CD8^{CD45.1} T cells in close proximity with Foxp3-expressing T_{REG} cells in Foxp3^{WT} mice, which were not readily observed in Foxp3^{ΔTbx21} animals despite similar CD8 T cell infiltration (FIG. 6g, FIG. 12c).

The requirement of CXCR3 to promote T_{RM} development suggests T_{REG} provide a short range acting or cell-bound effector molecule. Type 1 cytokines, such as IL-12, can maintain high levels of T-bet and Eomes, thereby preventing the differentiation of T_{RM} cells and the expression of CD103. IL12RB2-deficient CD8+ T cells have increased proportions of cells expressing CD103 and T cell clusters have higher TGFβ transcripts⁵⁹. IL-10 could reduce IL-12 expression and dendritic cell maturation⁶⁶. However, IL-10-deficient T_{REG} cells are able to assist in the efficient development of T_{RM} cells (FIG. 6h), and IL-10 deficient animals did not show a reduction in the T_{RM} cell compartment (FIG. 12d,e). Furthermore, EBI₃-deficient T_{REG} cells, unable to generate IL-35, similarly facilitated the generation of T_{RM} cells (FIG. 6i), nor was the T_{RM} cell compartment reduced in EBI3-deficient bone marrow chimeric animals (FIG. 12g,h).

Many cells, especially at the mucosal barrier, are able to make TGFβ⁶⁷. Yet, TGFβ is produced as an inactive precursor, which requires cleavage from its latency-associated peptide. TGF-β has potent cell modulation activity, acting on numerous immune and non-immune cell types, hence its availability is strictly regulated in the local microenvironment. It was recently shown that T_{REG} cells can activate TGFβ via the integrin αvβ8 and that this protein is upregulated in activated/effector T_{REG} cells, thereby reducing local bioactive TGFβ68, and the inventors hypothesised that specific recruitment of CXCR3-expressing T_{REG} cells, which do not show differential expression for TGFβ1 or Itgb8 under steady state (FIG. 5f, FIG. 12f)⁴², to sites with effector CD8 T cells may enable the local release of TGFβ. Similar to Foxp3^{ΔTbx21} mice, bone marrow chimeras generated from Foxp3^{Δltg88} mice showed a decrease in T_{RM} cells and an increase in KLRG1-expressing effector CD8+ T cells (FIG. 12g,i). Importantly, in the absence of Itgβ8 only in T_{REG} cells, there was inefficient promotion of T_{RM} cell generation (FIG. 6j). To understand if T_{REG}-derived TGFβ plays a deterministic role, the inventors used Foxp3^{ΔTgfβ1} mice. In line with Foxp3^{Δltg88} mice, the absence of TGFβ₁ from T_{REG} cells only resulted in increased effector T cells and reduced proportion of T_{RM} cells (FIG. 12g,j). Upon adoptive transfer, T_{REG} cells deficient in supplying TGFβ₁ could not rescue the development of T_{RM} cells in Foxp3^{ΔTbx21} mice (FIG. 6k). Collectively, and without wishing to be bound to any particular theory, the data shows that T_{REG} cells are recruited via T-bet-induced expression of CXCR3 produce TGFβ₁ and make it local bioavailability of via the expression of αvβ8 integrin to promote the development of T_{RM} cells in inflamed tissues.

### Discussion

The induction of long-lived cellular immunity in non-lymphoid tissues is important to protect against reinfection, as well as a major aim in vaccine design. The inventor's data supports a model in which CD8+ T cells home to tissues as effector cells or memory cell precursors, which subsequently differentiate into T_{RM} cells upon receiving local cues⁶⁹. T cell activation in SLOs induces the expression of a large variety of tissue homing receptors that guide activated T cells to non-lymphoid tissues ensuring that effector T cells inspect most peripheral tissues^{69, 70}. The unique profile of T_{RM} cells suggests that factors in the tissue microenvironment instruct the differentiation of effector cells into T_{RM} cells.

The inventor's data is based on localised infection models and a polyclonal TCR repertoire. Using localised infection models, it has been shown that optimal T_{RM} cell development, but not maintenance, requires inflammation-mediated trafficking and cognate antigen in the local microenvironment^{61, 71, 72}. Although this is in contrast with observations using systemic viral infections where IEL numbers remain stable⁶⁹, the inventor's observations are in line with previously reported small intestinal infection⁵⁸, and suggest this is a characteristic of local inflammation. Local cues, such as cytokines and secondary antigen encounter may be required for T_{RM} cell differentiation from recruited effector or memory precursor T cells. The inventor's data supports this model and extends it with the need to recruit T_{REG} cells to the site of inflammation and their ability to raise bioactive TGFβ levels that facilitate effector-to-memory development. Upon total T_{REG} cell depletion, numbers of T_{RM} cells in the central nervous system were reduced upon viral infection⁷³, suggesting a role of T_{REG} cells in the development or maintenance of T_{RM} cells. The inventors extend this observation by showing that local recruitment of type 1 T_{REG} cells is critical, whereupon expression of Itgβ8 promotes T_{RM} development, which critically relies on locally supplied TGFβ and its bioavailability^{23, 26, 59}. Local antigen can retain CD8+ T cells in tissues, which form stable contacts with infected cells and whereupon CD69 is re-expressed, a process that requires CXCR3 expression⁷⁴. Although in non-inflamed tissues the inventors report a reduction in CD4 and T_{REG} cells, CD4 T cells are recruited upon inflammation. Although the supplementation of T_{REG} cells is sufficient to enhance T_{RM} cell development, this does not exclude a supportive role of CD4 T cells. The inventors show that CXCR3 expression, also identifying type 1 T_{REG} cells in humans⁷⁵, is required to recruit T_{REG} cells to the site of inflammation providing a rational for the requirement of the upstream transcription factor T-bet. In the absence of CXCR3, T_{REG} cell tissue recruitment is limited, resulting in enhanced type 1 immunity and immunopathology^{42, 76}. Although the absence of T_{REG} cells is able to enhance immune responses, even resulting in sterile immunity, loss of subsequent immunity was reported⁵³. The inventors show here that type 1 T_{REG} cells provide TGFβ and make it available locally with the expression of αvβ8 integrin, thereby facilitating the development of T_{RM} cells, supporting life-long immune surveillance and increasing tissue protection against invading microorganisms.

The inventors observed reduced T_{RM} differentiation in several tissues assessed in the absence of T-bet-expressing T_{REG} cells. This highlights that specific tissue microenvironments do not play a critical role in the development of T_{RM} cells or the recruitment of type 1 T_{REG} cells. Nevertheless, tissue specific differences may alter the amplitude of T_{RM} development or their phenotype. T_{RM} cells in the intestine are known to predominantly produce IFNγ, while those in the epidermis have been shown to be able to produce IL-17 after microbial challenge. Furthermore, additional tissue insults can alter epidermal T_{RM} cell function, contributing to wound repair^{77,78}. The colon was the notable exception in which the inventors did not find alterations in T_{RM} or effector cells, nor were ratios in T cell subsets altered, in the absence of type 1 T_{REG} cells. Although T_{REG} cells require T-bet to express CXCR3 and to be recruited to inflammatory sites, T-bet does not seem to control CXCR3 expression in the colon⁴². The disjunction between T-bet and CXCR3 in the colon suggests alternative immune regulation in the organ harbouring the largest content of microbes.

TGFβ is a potent driver of CD103 expression on CD8+ T cells in vitro and in vivo²², and has been shown to reduce KLRG1 expression⁴³. Furthermore, the importance of reducing inflammation for T_{RM} cell development is suggested by diminished CD103-expression during chronic infection^{23,79}. In addition, TGFβRII-deficient CD8 T cells fail to become or remain T_{RM} cells^{19, 58}. In addition to CD103⁺ T_{RM} cells, a CD103⁻ T_{RM} cell population has been reported^{22, 58, 62}. The stability of this population may depend on tissue type and antigen persistence. In the inventor's models in control mice, looking at steady state under specific pathogen-free conditions, as well as after Ev challenge, CD103-T cells were a minor population. Instead, CD103-T cells observed in Foxp3^{ΔTbx21} mice expressed KLRG1 and high levels of Eomes, characteristics of T cells in a transition phase to express CD103 and switch off Eomes⁸⁰. Without wishing to be bound to any particular theory, the inventor's data does reveal an important role for type 1 T_{REG} cells in T_{RM} cell development, but a smaller T_{RM} cell population could still be generated, which suggests other cells may make an additional contribution in releasing TGFβ. Alternative sources of generation of bioactive TGFβ have been reported, including stromal epithelial cells, important for the maintenance of T_{RM} cells⁸¹.

T_{REG} cells are critical in dampening excessive immune responses, thereby preventing autoimmunity and immunopathology, and may reduce the amplitude of responses upon infection and vaccination as measured in blood. However, the inventor's data highlights their important role in efficiently generating tissue resident memory T cells from effector or memory precursors, which would otherwise become exhausted. T_{REG} cells thereby ensure that critical numbers of T cells are available for immunosurveillance in tissues to prevent or reduce re-infection as well as reducing pathogen load of new infections.

### Example 2

### Results

The inventors assessed whether the generation of T_{RM} cells was possible in the absence of T_{REG} cells in the medium, as detailed in FIG. 15.

Referring to FIG. 16, there is shown that the addition of TGFβ in cultures with IL-15, antigen presenting cells (BMDC) and previously activated CD8 T cells is sufficient to establish T_{RM} features such as continued expression of CD69 and CD103 in the absence of additional T_{REG} cells.

Referring to FIG. 17, there is shown that the combination of IL-15, TGFβ, antigen presenting cells and previously activated CD8 T cells with the addition of IL-7 can enhance the migratory capacity of generated T_{RM} cells based on their CTLA-4 expression profile (Front. Immunol., 27 Nov. 2018; Brunner-Weinzierl and Rudd; Kieke et al., PLOS One, 27/5/09)

Referring to FIG. 18, there is shown that the combination of IL-15, TGFβ, antigen presenting cells and previously activated CD8 T cells with the addition of IL-2 can enhance the migratory capacity of generated T_{RM} cells based on their CTLA-4 expression profile, but this is not as consistent as the addition of IL-7 in amount per cell or over biological repeats.

Furthermore, the inventors assessed if the cells produced in vitro maintained their therapeutic properties, in particular, their ability to migrate and survive in vivo inside the tissues, the experimental setup detailed in FIG. 19.

The inventors have recreated the in vivo conditions in an in vitro setup consisting of effector CD8+ T cells and bone marrow derived dendritic cells (BMDC). The T cells are stimulated in vitro and expanded in a similar manner to the produce a large amounts of cells for T cell therapies. The inventors show that the addition of bioactive TGFβ can replace the role of T_{REG} cells in the development of T cells resembling T_{RM} cells, with continued expression of the markers and tissue retention factors CD69 and CD103 (FIG. 20). CD69 is an activation marker normally transiently expressed upon T cell activation. CD69 is a C-type lectin, which are most likely involved in retention of T_{RM} cells in non-lymphoid tissues, including solid tumours⁹⁸. CD69 can form a complex with sphingosine-1-phosphate (S1P) 1, thereby preventing its binding to the S1P receptor that would trigger T cell egress out of tissues. Furthermore, the data show that the addition of IL-7 and IL-2 induces a strong CTLA-4 expression (FIGS. 17 and 18), which is linked with enhanced T cell migration^{99,100, 101}. Although CTLA-4 expression appears to be stronger with the addition of IL-2, replicate experiments have shown more consistency with the addition of IL-7 than with the addition of IL-2. Moreover, adding IL-7 resulted in individual cells having stronger CTLA-4 expression.

The data resulting from transferring the in vitro generated and expanded T cells in vivo show that effector cells, those generated in the absence of TGFβ are not found in substantial numbers in the tissues (FIGS. 21, 22, 23 and 24). T_{RM} cells on the other hand, especially when stimulated with IL-7 are readily found 40 days post-transfer including in all organs tested such as the lungs, liver and lamina propria and intra-epithelial compartments of the small intestine (FIGS. 21, 22, 23 and 24).

### SUMMARY

In summary, the inventors have developed a highly novel and innovative protocol to generate T cells (known as T_{RM} cells) that are able to deeply penetrate tumours (and especially solid tumours) to contribute to a step-change in T cell therapies against tumours or infections. The inventor's protocol results in vitro generated and expanded cells with the phenotype of migratory and tissue penetrating cells based on the expression of CTLA-4, CD69 and CD103. Conform their phenotype and in contrast to effector T cells, the generated cells are readily found in a variety of lymphoid and non-lymphoid tissues at least 40 days after adoptive transfer into a full mouse host. The inventors' work will make important inroads for efficacious treatment of organ infections and for those cancer patients suffering from solid tumours, which are much harder to treat. However, the inventors believe that the tissue-penetrating ability of the T_{RM} cells will go beyond the targeting of infections or primary tumours and may provide critical organ-wide immunosurveillance directed against metastasis that have migrated to, often less accessible, tissues away from the primary tumour.

### REFERENCES

1. Stary, G. et al. VACCINES. A mucosal vaccine against Chlamydia trachomatis generates two waves of protective memory T cells. Science 348, aaa8205 (2015).
2. Masopust, D., Vezys, V., Marzo, A. L. & Lefrancois, L. Preferential localization of effector memory cells in nonlymphoid tissue. Science 291, 2413-2417. (2001).
3. Reinhardt, R. L., Khoruts, A., Merica, R., Zell, T. & Jenkins, M. K. Visualizing the generation of memory CD4 T cells in the whole body. Nature 410, 101-105. (2001).
4. Mueller, S. N. & Mackay, L. K. Tissue-resident memory T cells: local specialists in immune defence. Nature reviews. Immunology 16, 79-89 (2016).
5. Konjar, S., Ferreira, C., Blankenhaus, B. & Veldhoen, M. Intestinal Barrier Interactions with Specialized CD8 T Cells. Front Immunol 8, 1281 (2017).
6. Ariotti, S. et al. Tissue-resident memory CD8+ T cells continuously patrol skin epithelia to quickly recognize local antigen. Proceedings of the National Academy of Sciences of the United States of America 109, 19739-19744 (2012).
7. Schenkel, J. M., Fraser, K. A., Vezys, V. & Masopust, D. Sensing and alarm function of resident memory CD8(+) T cells. Nature immunology 14, 509-513 (2013).
8. Slutter, B., Pewe, L. L., Kaech, S. M. & Harty, J. T. Lung airway-surveilling CXCR3 (hi) memory CD8(+) T cells are critical for protection against influenza A virus. Immunity 39, 939-948 (2013).
9. Wu, T. et al. Lung-resident memory CD8 T cells (TRM) are indispensable for optimal cross-protection against pulmonary virus infection. J Leukoc Biol 95, 215-224 (2014).
10. Konjar, S. et al. Mitochondria maintain controlled activation state of epithelial-resident T lymphocytes. Sci Immunol 3, eaan2543 (2018).
11. Intlekofer, A. M. et al. Effector and memory CD8+ T cell fate coupled by T-bet and eomesodermin. Nature immunology 6, 1236-1244 (2005).
12. Pearce, E. L. et al. Control of effector CD8+ T cell function by the transcription factor Eomesodermin. Science 302, 1041-1043 (2003).
13. Banerjee, A. et al. Cutting edge: The transcription factor eomesodermin enables CD8+ T cells to compete for the memory cell niche. Journal of immunology 185, 4988-4992 (2010).
14. Intlekofer, A. M. et al. Requirement for T-bet in the aberrant differentiation of unhelped memory CD8+ T cells. The Journal of experimental medicine 204, 2015-2021 (2007).
15. Joshi, N. S. et al. Inflammation directs memory precursor and short-lived effector CD8(+) T cell fates via the graded expression of T-bet transcription factor. Immunity 27, 281-295 (2007).
16. Pipkin, M. E. et al. Interleukin-2 and inflammation induce distinct transcriptional programs that promote the differentiation of effector cytolytic T cells. Immunity 32, 79-90 (2010).
17. Joshi, N. S. et al. Increased numbers of preexisting memory CD8 T cells and decreased T-bet expression can restrain terminal differentiation of secondary effector and memory CD8 T cells. Journal of immunology 187, 4068-4076 (2011).
18. Kaech, S. M. & Ahmed, R. Memory CD8+ T cell differentiation: initial antigen encounter triggers a developmental program in naive cells. Nature immunology 2, 415-422. (2001).
19. Mackay, L. K. et al. The developmental pathway for CD103 (+) CD8+tissue-resident memory T cells of skin. Nature immunology 14, 1294-1301 (2013).
20. Wakim, L. M. et al. The molecular signature of tissue resident memory CD8 T cells isolated from the brain. Journal of immunology 189, 3462-3471 (2012).
21. Nakayamada, S. et al. Early Th1 cell differentiation is marked by a Tfh cell-like transition. Immunity 35, 919-931 (2011).
22. Casey, K. A. et al. Antigen-independent differentiation and maintenance of effector-like resident memory T cells in tissues. Journal of immunology 188, 4866-4875 (2012).
23. Zhang, N. & Bevan, M. J. Transforming growth factor-beta signaling controls the formation and maintenance of gut-resident memory T cells by regulating migration and retention. Immunity 39, 687-696 (2013).
24. Sheridan, B. S. et al. Oral infection drives a distinct population of intestinal resident memory CD8(+) T cells with enhanced protective function. Immunity 40, 747-757 (2014).
25. Laidlaw, B. J. et al. CD4+ T cell help guides formation of CD103+ lung-resident memory CD8+ T cells during influenza viral infection. Immunity 41, 633-645 (2014).
26. Mackay, L. K. et al. T-box Transcription Factors Combine with the Cytokines TGF-beta and IL-15 to Control Tissue-Resident Memory T Cell Fate. Immunity 43, 1101-1111 (2015).
27. Laidlaw, B. J., Craft, J. E. & Kaech, S. M. The multifaceted role of CD4(+) T cells in CD8(+) T cell memory. Nature reviews. Immunology 16, 102-111 (2016).
28. Feau, S., Arens, R., Togher, S. & Schoenberger, S. P. Autocrine IL-2 is required for secondary population expansion of CD8(+) memory T cells. Nature immunology 12, 908-913 (2011).
29. Yu, F., Sharma, S., Edwards, J., Feigenbaum, L. & Zhu, J. Dynamic expression of transcription factors T-bet and GATA-3 by regulatory T cells maintains immunotolerance. Nature immunology 16, 197-206 (2015).
30. McNally, A., Hill, G. R., Sparwasser, T., Thomas, R. & Steptoe, R. J. CD4+CD25+ regulatory T cells control CD8+ T-cell effector differentiation by modulating IL-2 homeostasis. Proceedings of the National Academy of Sciences of the United States of America 108, 7529-7534 (2011).
31. de Goer de Herve, M. G., Jaafoura, S., Vallee, M. & Taoufik, Y. FoxP3(+) regulatory CD4 T cells control the generation of functional CD8 memory. Nature communications 3, 986 (2012).
32. Pace, L. et al. Regulatory T cells increase the avidity of primary CD8+ T cell responses and promote memory. Science 338, 532-536 (2012).
33. Laidlaw, B. J. et al. Production of IL-10 by CD4(+) regulatory T cells during the resolution of infection promotes the maturation of memory CD8(+) T cells. Nature immunology 16, 871-879 (2015).
34. Kalia, V., Penny, L. A., Yuzefpolskiy, Y., Baumann, F. M. & Sarkar, S. Quiescence of Memory CD8(+) T Cells Is Mediated by Regulatory T Cells through Inhibitory Receptor CTLA-4. Immunity 42, 1116-1129 (2015).
35. Koch, M. A. et al. The transcription factor T-bet controls regulatory T cell homeostasis and function during type 1 inflammation. Nature immunology 10, 595-602 (2009).
36. Hall, A. O. et al. The cytokines interleukin 27 and interferon-gamma promote distinct Treg cell populations required to limit infection-induced pathology. Immunity 37, 511-523 (2012).
37. Reis, B. S., Hoytema van Konijnenburg, D. P., Grivennikov, S. I. & Mucida, D. Transcription factor T-bet regulates intraepithelial lymphocyte functional maturation. Immunity 41, 244-256 (2014).
38. Lord, G. M. et al. T-bet is required for optimal proinflammatory CD4+ T-cell trafficking. Blood 106, 3432-3439 (2005).
39. Levine, A. G. et al. Stability and function of regulatory T cells expressing the transcription factor T-bet. Nature 546, 421-425 (2017).
40. Obar, J. J. & Lefrancois, L. Memory CD8+ T cell differentiation. Ann N Y Acad Sci 1183, 251-266 (2010).
41. Omilusik, K. D. et al. Sustained Id2 regulation of E proteins is required for terminal differentiation of effector CD8(+) T cells. The Journal of experimental medicine 215, 773-783 (2018).
42. Tan, T. G., Mathis, D. & Benoist, C. Singular role for T-BET+CXCR3+ regulatory T cells in protection from autoimmune diabetes. Proceedings of the National Academy of Sciences of the United States of America 113, 14103-14108 (2016).
43. Schwartzkopff, S. et al. TGF-beta downregulates KLRG1 expression in mouse and human CD8(+) T cells. European journal of immunology 45, 2212-2217 (2015).
44. Janssen, E. M. et al. CD4+ T-cell help controls CD8+ T-cell memory via TRAIL-mediated activation-induced cell death. Nature 434, 88-93 (2005).
45. Oh, S. et al. IL-15 as a mediator of CD4+help for CD8+ T cell longevity and avoidance of TRAIL-mediated apoptosis. Proceedings of the National Academy of Sciences of the United States of America 105, 5201-5206 (2008).
46. Madakamutil, L. T. et al. CD8alphaalpha-mediated survival and differentiation of CD8 memory T cell precursors. Science 304, 590-593 (2004).
47. Lertmemongkolchai, G., Cai, G., Hunter, C. A. & Bancroft, G. J. Bystander activation of CD8+ T cells contributes to the rapid production of IFN-gamma in response to bacterial pathogens. Journal of immunology 166, 1097-1105 (2001).
48. Chu, T. et al. Bystander-activated memory CD8 T cells control early pathogen load in an innate-like, NKG2D-dependent manner. Cell reports 3, 701-708 (2013).
49. Fernandez-Ruiz, D. et al. Liver-Resident Memory CD8(+) T Cells Form a Front-Line Defense against Malaria Liver-Stage Infection. Immunity 45, 889-902 (2016).
50. Herndler-Brandstetter, D. et al. KLRG1(+) Effector CD8(+) T Cells Lose KLRG1, Differentiate into All Memory T Cell Lineages, and Convey Enhanced Protective Immunity. Immunity 48, 716-729 e718 (2018).
51. Rose, M. E., Joysey, H. S., Hesketh, P., Grencis, R. K. & Wakelin, D. Mediation of immunity to Eimeria vermiformis in mice by L3T4+ T cells. Infection and immunity 56, 1760-1765 (1988).
52. Rose, M. E., Wakelin, D. & Hesketh, P. Gamma interferon controls Eimeria vermiformis primary infection in BALB/c mice. Infection and immunity 57, 1599-1603 (1989).
53. Belkaid, Y., Piccirillo, C. A., Mendez, S., Shevach, E. M. & Sacks, D. L. CD4+CD25+ regulatory T cells control Leishmania major persistence and immunity. Nature 420, 502-507 (2002).
54. Ramsburg, E., Tigelaar, R., Craft, J. & Hayday, A. Age-dependent requirement for gammadelta T cells in the primary but not secondary protective immune response against an intestinal parasite. The Journal of experimental medicine 198, 1403-1414 (2003).
55. Miragaia, R. J. et al. Single-Cell Transcriptomics of Regulatory T Cells Reveals Trajectories of

Tissue Adaptation. Immunity 50, 493-504 e497 (2019).
56. Zemmour, D. et al. Single-cell gene expression reveals a landscape of regulatory T cell phenotypes shaped by the TCR. Nature immunology 19, 291-301 (2018).
57. Xiong, Y., Ahmad, S., Iwami, D., Brinkman, C. C. & Bromberg, J. S. T-bet Regulates Natural Regulatory T Cell Afferent Lymphatic Migration and Suppressive Function. Journal of immunology 196, 2526-2540 (2016).
58. Bergsbaken, T. & Bevan, M. J. Proinflammatory microenvironments within the intestine regulate the differentiation of tissue-resident CD8(+) T cells responding to infection. Nature immunology 16, 406-414 (2015).
59. Bergsbaken, T., Bevan, M. J. & Fink, P. J. Local Inflammatory Cues Regulate Differentiation and Persistence of CD8(+) Tissue-Resident Memory T Cells. Cell reports 19, 114-124 (2017).
60. Harris, T. H. et al. Generalized Levy walks and the role of chemokines in migration of effector CD8+ T cells. Nature 486, 545-548 (2012).
61. Khan, T. N., Mooster, J. L., Kilgore, A. M., Osborn, J. F. & Nolz, J. C. Local antigen in nonlymphoid tissue promotes resident memory CD8+ T cell formation during viral infection. The Journal of experimental medicine 213, 951-966 (2016).
62. Wakim, L. M., Woodward-Davis, A. & Bevan, M. J. Memory T cells persisting within the brain after local infection show functional adaptations to their tissue of residence. Proceedings of the National Academy of Sciences of the United States of America 107, 17872-17879 (2010).
63. Iijima, N. & Iwasaki, A. T cell memory. A local macrophage chemokine network sustains protective tissue-resident memory CD4 T cells. Science 346, 93-98 (2014).
64. Natsuaki, Y. et al. Perivascular leukocyte clusters are essential for efficient activation of effector T cells in the skin. Nature immunology 15, 1064-1069 (2014).
65. Collins, N. et al. Skin CD4(+) memory T cells exhibit combined cluster-mediated retention and equilibration with the circulation. Nature communications 7, 11514 (2016).
66. Corinti, S., Albanesi, C., la Sala, A., Pastore, S. & Girolomoni, G. Regulatory activity of autocrine IL-10 on dendritic cell functions. Journal of immunology 166, 4312-4318 (2001).
67. Li, M. O., Wan, Y. Y., Sanjabi, S., Robertson, A. K. & Flavell, R. A. Transforming growth factor-beta regulation of immune responses. Annual review of immunology 24, 99-146 (2006).
68. Worthington, J. J. et al. Integrin alphavbeta8-Mediated TGF-beta Activation by Effector Regulatory T Cells Is Essential for Suppression of T-Cell-Mediated Inflammation. Immunity 42, 903-915 (2015).
69. Masopust, D. et al. Dynamic T cell migration program provides resident memory within intestinal epithelium. The Journal of experimental medicine 207, 553-564 (2010).
70. Butcher, E. C. & Picker, L. J. Lymphocyte homing and homeostasis. Science 272, 60-66. (1996).
71. Gebhardt, T. et al. Memory T cells in nonlymphoid tissue that provide enhanced local immunity during infection with herpes simplex virus. Nature immunology 10, 524-530 (2009).
72. Mackay, L. K. et al. Long-lived epithelial immunity by tissue-resident memory T (T_{RM}) cells in the absence of persisting local antigen presentation. Proceedings of the National Academy of Sciences of the United States of America 109, 7037-7042 (2012).
73. Graham, J. B., Da Costa, A. & Lund, J. M. Regulatory T cells shape the resident memory T cell response to virus infection in the tissues. Journal of immunology 192, 683-690 (2014).
74. Hickman, H. D. et al. CXCR3 chemokine receptor enables local CD8(+) T cell migration for the destruction of virus-infected cells. Immunity 42, 524-537 (2015).
75. Duhen, T., Duhen, R., Lanzavecchia, A., Sallusto, F. & Campbell, D. J. Functionally distinct subsets of human FOXP3+ Treg cells that phenotypically mirror effector Th cells. Blood 119, 4430-4440 (2012).
76. Paust, H. J. et al. CXCR3+ Regulatory T Cells Control TH1 Responses in Crescentic GN. J Am Soc Nephrol 27, 1933-1942 (2016).
77. Harrison, O. J. et al. Commensal-specific T cell plasticity promotes rapid tissue adaptation to injury. Science 363 (2019).
78. Ariotti, S. & Veldhoen, M. Immunology: Skin T Cells Switch Identity to Protect and Heal. Curr Biol 29, R220-R223 (2019).
79. Beura, L. K. et al. Lymphocytic choriomeningitis virus persistence promotes effector-like memory differentiation and enhances mucosal T cell distribution. J Leukoc Biol 97, 217-225 (2015).
80. Mueller, S. N., Gebhardt, T., Carbone, F. R. & Heath, W. R. Memory T cell subsets, migration patterns, and tissue residence. Annual review of immunology 31, 137-161 (2013).
81. Mohammed, J. et al. Stromal cells control the epithelial residence of DCs and memory T cells by regulated activation of TGF-beta. Nature immunology 17, 414-421 (2016).
82. Intlekofer, A. M. et al. Anomalous type 17 response to viral infection by CD8+ T cells lacking T-bet and eomesodermin. Science 321, 408-411 (2008).
83. Rubtsov, Y. P. et al. Regulatory T cell-derived interleukin-10 limits inflammation at environmental interfaces. Immunity 28, 546-558 (2008).
84. Luche, H., Weber, O., Nageswara Rao, T., Blum, C. & Fehling, H. J. Faithful activation of an extra-bright red fluorescent protein in “knock-in” Cre-reporter mice ideally suited for lineage tracing studies. European journal of immunology 37, 43-53 (2007).
85. Hancock, W. W. et al. Requirement of the chemokine receptor CXCR3 for acute allograft rejection.

The Journal of experimental medicine 192, 1515-1520 (2000).
86. Kuhn, R., Lohler, J., Rennick, D., Rajewsky, K. & Muller, W. Interleukin-10-deficient mice develop chronic enterocolitis. Cell 75, 263-274 (1993).
87. Nieuwenhuis, E. E. et al. Disruption of T helper 2-immune responses in Epstein-Barr virus-induced gene 3-deficient mice. Proceedings of the National Academy of Sciences of the United States of America 99, 16951-16956 (2002).
88. Travis, M. A. et al. Loss of integrin alpha(v)beta8 on dendritic cells causes autoimmunity and colitis in mice. Nature 449, 361-365 (2007).
89. Azhar, M. et al. Generation of mice with a conditional allele for transforming growth factor beta 1 gene. Genesis 47, 423-431 (2009).
90. Li, Y. et al. Exogenous stimuli maintain intraepithelial lymphocytes via aryl hydrocarbon receptor activation. Cell 147, 629-640 (2011).
91. Figueiredo-Campos, P., Ferreira, C., Blankenhaus, B. & Veldhoen, M. Eimeria vermiformis Infection Model of Murine Small Intestine. Bio-Protocol 8 (2018).
92. Cossarizza, A. et al. Guidelines for the use of flow cytometry and cell sorting in immunological studies (second edition). Eur J Immunol 49, 1457-1973 (2019).
93. Butler, A., Hoffman, P., Smibert, P., Papalexi, E. & Satija, R. Integrating single-cell transcriptomic data across different conditions, technologies, and species. Nat Biotechnol 36, 411-420 (2018).
94. Savas, P., Virassamy, B., Ye, C. et al. Single-cell profiling of breast cancer T cells reveals a tissue-resident memory subset associated with improved prognosis. Nat Med 24, 986-993 (2018).
95. Zhi-Qiang Wang, Katy Milne, Heather Derocher, John R. Webb, Brad H. Nelson and Peter

H. Watson. CD103 and Intratumoral Immune Response in Breast Cancer. Clin Cancer Res (22) (24) 6290-6297 (2018).
96. John R. Webb, Katy Milne, Peter Watson, Ronald J. deLeeuw and Brad H. Nelson. Tumor-Infiltrating Lymphocytes Expressing the Tissue Resident Memory Marker CD103 Are Associated with Increased Survival in High-Grade Serous Ovarian Cancer. Clin Cancer Res (20) (2) 434-444 (2014).
97. Jarem Edwards et al. CD103⁺ Tumor-Resident CD8⁺ T Cells Are Associated with Improved Survival in Immunotherapy-Naïve Melanoma Patients and Expand Significantly During Anti-PD-1 Treatment. Clin Cancer Res (24) (13) 3036-3045 (2018).
98. Mami-Chonaib P. Blane C, Corgnac S. et al. Resident memory T cells, critical components in tumor immunology. Journal for ImmunoTherapy of Cancer 6:87 2018.
99. Biliana Lozanoska-Ochser, Nigel J. Klein, Guo C. Huang, Raymond A. Alvarez and Mark Peakman. Expression of CD86 on Human Islet Endothelial Cells Facilitates T Cell Adhesion and Migration. J Immunol. 181 (9) 6109-6116 (2008),
100. Knieke K, Hoff H, Maszyna F, Kolar P. Schrage A, Hamann A, et al. (2009) CD152 (CTLA-4) Determines CD4 T Cell Migration In Vitro and In Vivo. PLOS ONE 4 (5): 5702.
101. Brunner-Weinzierl Monika C., Rudd Christopher E. CTLA-4 and PD-1 Control of T-Cell Motility and Migration: Implications for Tumor Immunotherapy. JOURNAL=Frontiers in Immunology

1. A method for producing a tissue-resident memory T cell (T_{RM}), the method comprising culturing a lymphocyte in the presence of transforming growth factor beta (TGFβ) and/or co-culturing the lymphocyte with a regulatory T cell.

2. The method according to claim 1, wherein the lymphocyte is cultured in the presence of TGFβ, preferably wherein the lymphocyte is not co-cultured with a regulatory T cell.

3. The method according to claim 1, wherein the lymphocyte is a naïve, effector or memory CD8+ T lymphocyte.

4. The method according to claim 1, wherein the TGF□ _{□}is present at a concentration of between 0.01 ng/ml and 50 ng/ml.

5. The method according to claim 1, wherein the lymphocyte has been obtained from tissue of a human or non-human animal, optionally wherein the tissue may be selected from the group consisting of: blood, spleen, lymph node, lung, gastrointestinal tract, skin, prostate mammary gland tissue, liver, bone marrow and pancreas.

6. The method according to claim 1, wherein the T_{RM} is characterised by expression of cluster of differentiation 8 (CD8), cluster of differentiation 69 (CD69), Hobit, aryl hydrocarbon receptor (AhR) and/or cluster of differentiation 103 (CD103).

7. The method according to claim 1, wherein the T_{RM} is characterised by the absence of expression of killer cell lectin-like receptor subfamily G member (KLRG1) and/or Eomesodermin (Eomes).

8. The method according to claim 1, wherein the method comprises culturing the lymphocyte in the presence of IL-2, IL-4, IL-7, IL-12, IL-15 and/or IL-21, optionally, wherein the method further comprises comprising culturing the lymphocyte in the presence of interleukin 33 (IL-33) and/or at least one interleukin 1 family member, optionally wherein the at least one interleukin 1 family member is IL-1a, IL-1b and/or IL-18.

9-14. (canceled)

15. The method according to claim 1, wherein the lymphocyte is cultured in a culture media comprising at least one aryl hydrocarbon receptor (AhR) ligand.

16. The method according to claim 9, wherein the AhR ligand is selected from a halogenated aromatic hydrocarbon, a polycyclic aromatic hydrocarbon, a dietary derived aryl hydrocarbon, a heme metabolite, an indigoid, StemRegenin 1 and a tryptophan metabolite.

17. The method according to claim 1, wherein the lymphocyte is cultured in a culture media comprising at least one lipid.

18. The method according to claim 1, wherein the lymphocyte is cultured in a culture media comprising an antigen, optionally wherein the antigen is a tumour antigen.

19. The method according to claim 1, wherein the regulatory T cell is characterised by expression of Foxp3, or is absent.

20. The method according to claim 1, wherein the method further comprises culturing the lymphocyte with a dendritic cell.

21. The method according to claim 1, wherein the method further comprises expanding a population of tissue-resident memory T cells (T_{RM}).

22. The method according to claim 15, wherein the method further comprises culturing the T_{RM} cells in the presence of IL-2, IL-4, IL-7, IL-12, IL-15 and/or IL-21.

23. A tissue-resident memory T cell (T_{RM}) derived from a lymphocyte cultured in the presence of transforming growth factor beta (TGFβ) and/or co-cultured the with a regulatory T cell.

24-25. (canceled)

26. A method of preventing, treating or ameliorating of cancer or an infection in a subject in need thereof, the method comprising administering a tissue-resident memory T cell derived from a lymphocyte cultured in the presence of transforming growth factor beta (TGFβ) and/or co-cultured the with a regulatory T cell, to the subject.

27. The tissue-resident memory T cell (T_{RM}) according to claim 23 or an expanded population thereof, wherein the tissue-resident memory T cell (T_{RM}) or the expanded population thereof is comprised in a pharmaceutical composition, optionally wherein the pharmaceutical composition further comprises a pharmaceutically acceptable excipient.

28. (canceled)


SEQUENCE LISTING (28 sequences)

SEQ ID NO: 1 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 3 - protein, 199 aa, Homo sapiens
MSSENCFVAENSSLHPESGQENDATSPHFSTRHEGSFQVPVLCAVMNVVFITILIIALIALSVGQYNCPGQYTFSMPSDSHVSSCSEDWVGYQRKCYFISTVKRSWTSAQNACSEHGATLAVIDSEKDMNFLKRYAGREEHWVGLKKEPGHPWKWSNGKEFNNWFNVTGSDKCVFLKNTEVSSMECEKNLYWICNKPYK

SEQ ID NO: 4 - protein, 1179 aa, Homo sapiens
MWLFHTLLCIASLALLAAFNVDVARPWLTPKGGAPFVLSSLLHQDPSTNQTWLLVTSPRTKRTPGPLHRCSLVQDEILCHPVEHVPIPKGRHRGVTVVRSHHGVLICIQVLVRRPHSLSSELTGTCSLLGPDLRPQAQANFFDLENLLDPDARVDTGDCYSNKEGGGEDDVNTARQRRALEKEEEEDKEEEEDEEEEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIISMEGTVGDALHYQLAQIGFSAQILDERQVLLGAVGAFDWSGGALLYDTRSRRGRFLNQTAAAAADAEAAQYSYLGYAVAVLHKTCSLSYIAGAPRYKHHGAVFELQKEGREASFLPVLEGEQMGSYFGSELCPVDIDMDGSTDFLLVAAPFYHVHGEEGRVYVYRLSEQDGSFSLARILSGHPGFTNARFGFAMAAMGDLSQDKLTDVAIGAPLEGFGADDGASFGSVYIYNGHWDGLSASPSQRIRASTVAPGLQYFGMSMAGGFDISGDGLADITVGTLGQAVVFRSRPVVRLKVSMAFTPSALPIGFNGVVNVRLCFEISSVTTASESGLREALLNFTLDVDVGKQRRRLQCSDVRSCLGCLREWSSGSQLCEDLLLMPTEGELCEEDCFSNASVKVSYQLQTPEGQTDHPQPILDRYTEPFAIFQLPYEKACKNKLFCVAELQLATTVSQQELVVGLTKELTLNINLTNSGEDSYMTSMALNYPRNLQLKRMQKPPSPNIQCDDPQPVASVLIMNCRIGHPVLKRSSAHVSVVWQLEENAFPNRTADITVTVTNSNERRSLANETHTLQFRHGFVAVLSKPSIMYVNTGQGLSHHKEFLFHVHGENLFGAEYQLQICVPTKLRGLQVVAVKKLTRTQASTVCTWSQERACAYSSVQHVEEWHSVSCVIASDKENVTVAAEISWDHSEELLKDVTELQILGEISFNKSLYEGLNAENHRTKITVVFLKDEKYHSLPIIIKGSVGGLLVLIVILVILFKCGFFKRKYQQLNLESIRKAQLKSENLLEEEN

SEQ ID NO: 5 - protein, 195 aa, Homo sapiens
MTDSVIYSMLELPTATQAQNDYGPQQKSSSSRPSCSCLVAIALGLLTAVLLSVLLYQWILCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKCPFADQALF

SEQ ID NO: 6 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 7 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 9 - protein, 524 aa, Homo sapiens
MKEESAAQLGCCHRPMALGGTGGSLSPSLDFQLFRGDQVFSACRPLPDMVDAHGPSCASWLCPLPLAPGRSALLACLQDLDLNLCTPQPAPLGTDLQGLQEDALSMKHEPPGLQASSTDDKKFTVKYPQNKDKLGKQPERAGEGAPCPAFSSHNSSSPPPLQNRKSPSPLAFCPCPPVNSISKELPFLLHAFYPGYPLLLPPPHLFTYGALPSDQCPHLLMLPQDPSYPTMAMPSLLMMVNELGHPSARWETLLPYPGAFQASGQALPSQARNPGAGAAPTDSPGLERGGMASPAKRVPLSSQTGTAALPYPLKKKNGKILYECNICGKSFGQLSNLKVHLRVHSGERPFQCALCQKSFTQLAHLQKHHLVHTGERPHKCSIPWVPGRNHWKSFQAWREREVCHKRFSSSSNLKTHLRLHSGARPFQCSVCRSRFTQHIHLKLHHRLHAPQPCGLVHTQLPLASLACLAQWHQGALDLMAVASEKHMGYDIDEVKVSSTSQGKARAVSLSSAGTPLVMGQDQNN

SEQ ID NO: 10 - protein, 848 aa, Homo sapiens
MNSSSANITYASRKRRKPVQKTVKPIPAEGIKSNPSKRHRDRLNTELDRLASLLPFPQDVINKLDKLSVLRLSVSYLRAKSFFDVALKSSPTERNGGQDNCRAANFREGLNLQEGEFLLQALNGFVLVVTTDALVFYASSTIQDYLGFQQSDVIHQSVYELIHTEDRAEFQRQLHWALNPSQCTESGQGIEEATGLPQTVVCYNPDQIPPENSPLMERCFICRLRCLLDNSSGFLAMNFQGKLKYLHGQKKKGKDGSILPPQLALFAIATPLQPPSILEIRTKNFIFRTKHKLDFTPIGCDAKGRIVLGYTEAELCTRGSGYQFIHAADMLYCAESHIRMIKTGESGMIVFRLLTKNNRWTWVQSNARLLYKNGRPDYIIVTQRPLTDEEGTEHLRKRNTKLPFMFTTGEAVLYEATNPFPAIMDPLPLRTKNGTSGKDSATTSTLSKDSLNPSSLLAAMMQQDESIYLYPASSTSSTAPFENNFFNESMNECRNWQDNTAPMGNDTILKHEQIDQPQDVNSFAGGHPGLFQDSKNSDLYSIMKNLGIDFEDIRHMQNEKFFRNDFSGEVDFRDIDLTDEILTYVQDSLSKSPFIPSDYQQQQSLALNSSCMVQEHLHLEQQQQHHQKQVVVEPQQQLCQKMKHMQVNGMFENWNSNQFVPFNCPQQDPQQYNVFTDLHGISQEFPYKSEMDSMPYTQNFISCNQPVLPQHSKCTELDYPMGSFEPSPYPTTSSLEDFVTCLQLPENQKHGLNPQSAIITPQTCYAGAVSMYQCQPEPQHTHVGQMQYNPVLPGQQAFLNKFQNGVLNETYPAELNNINNTQTTTHLQPLHHPSEARPFPDLTSSGFL

SEQ ID NO: 11 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 12 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 13 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 14 - protein, 414 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 15 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 16 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 17 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 18 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 19 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 20 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 21 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 22 - protein, 385 aa, Homo sapiens
MGCRRTREGPSKAMIFPWKCQSTQRDLWNIFKLWGWTMLCCDFLAHHGTDCWTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYNPLFIPVAVMVTAFSGLAFIIWLARRLKKGKKSKRSMNDPY

SEQ ID NO: 23 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 24 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 25 - protein, 415 aa, Homo sapiens
MELRKYGPGRLAGTVIGGAAQSKSQTKSDSITKEFLPGLYTAPSSPFPPSQVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 26 - protein, 769 aa, Homo sapiens
MCGSALAFFTAAFVCLQNDRRGPASFLWAAWVFSLVLGLGQGEDNRCASSNAASCARCLALGPECGWCVQEDFISGGSRSERCDIVSNLISKGCSVDSIEYPSVHVIIPTENEINTQVTPGEVSIQLRPGAEANFMLKVHPLKKYPVDLYYLVDVSASMHNNIEKLNSVGNDLSRKMAFFSRDFRLGFGSYVDKTVSPYISIHPERIHNQCSDYNLDCMPPHGYIHVLSLTENITEFEKAVHRQKISGNIDTPEGGFDAMLQAAVCESHIGWRKEAKRLLLVMTDQTSHLALDSKLAGIVVPNDGNCHLKNNVYVKSTTMEHPSLGQLSEKLIDNNINVIFAVQGKQFHWYKDLLPLLPGTIAGEIESKAANLNNLVVEAYQKLISEVKVQVENQVQGIYFNITAICPDGSRKPGMEGCRNVTSNDEVLFNVTVTMKKCDVTGGKNYAIIKPIGFNETAKIHIHRNCSCQCEDNRGPKGKCVDETFLDSKCFQCDENKCHFDEDQFSSESCKSHKDQPVCSGRGVCVCGKCSCHKIKLGKVYGKYCEKDDFSCPYHHGNLCAGHGECEAGRCQCFSGWEGDRCQCPSAAAQHCVNSKGQVCSGRGTCVCGRCECTDPRSIGRFCEHCPTCYTACKENWNCMQCLHPHNLSQAILDQCKTSCALMEQQHYVDQTSECFSSPSYLRIFFIIFIVTFLIGLLKVLIIRQVILQWNSNKIKSSSDYRVSASKKDKLILQSVCTRAVTYRREKPEEIKMDISKLNAHETFRCNF

SEQ ID NO: 27 - protein, 1048 aa, Homo sapiens
MAFPPRRRLRLGPRGLPLLLSGLLLPLCRAFNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET

SEQ ID NO: 28 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH